(19) United States
(12) Patent Application Publication
(10) Pub. No.: US 2005/0019766 A1
(43) Pub. Date: Jan. 27, 2005

(54) Isp-1 and ctb-1 genes and uses thereof

(76) Inventors: Jinliu Feng, Toronto (CA); Frederic Bussiere, Quebec (CA); Siegfried Hekimi, Montreal (CA)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/484,978

(22) PCT Filed: Jul. 26, 2002
(86) PCT No.: PCT/CA02/01165

Related U.S. Application Data
(60) Provisional application No. 60/307,840, filed on Jul. 27, 2001.

(51) Int. Cl. C12Q 1/68; C07H 21/04; C12N 9/64

(52) U.S. Cl. 435/6; 435/69.1; 435/320.1; 435/226; 435/325; 536/23.2

(57) ABSTRACT

The present invention relates to the characterization of mutants in the genes isp-1 and ctb-1, which are genes that have a function at the level of cellular physiology, mitochondrial respiration and electron transport, and resistance to oxidative stress, as well as regulating developmental, behavioral, reproductive and aging rates. Mutations in the genes isp-1 and ctb-1 are also provided. These genes and the protein they encode are used in a perspective of growth and aging control and in a therapeutic perspective for diseases in which mitochondrial function is altered. There is also provided methods of identification of compounds for the manipulation of the function of the isp-1 and ctb-1 genes and their protein products, as well as for the manipulation of the functions of mitochondria.

### TECHNICAL FIELD

The present invention relates to the identification of two genes: the gene isp-1 and the gene ctb-1. The invention discloses the use of the genes isp-1 and ctb-1 and their respective proteins ISP-1 and CTB-1, analogs and derivatives thereof to regulate the production of reactive oxygen species, as well as the timing of development and behavior, and determine life span.

### BACKGROUND OF THE INVENTION

In recent years the use of model systems to study the genetics of aging has become a prominent approach to understand the molecular mechanisms of aging. One choice organism for such studies has been the nematode C. elegans. The worm is practical because it is small, easily cultured, and short-lived. This has led to the isolation of mutations that dramatically lengthen life span. The great power of such mutants for the study of aging comes from the conclusion that the normal activity of a gene must limit the life span of the wild type when a loss-of-function mutation of the gene results in an increased life span of the mutant.

Genes and mutations that affect the life span of the worm can be grouped into three classes: genes that affect the dauer formation pathway (daf genes and others), genes that affect physiological rates (clk genes), and genes that are required for normal food intake (eat genes). Some other loci, of course, do not fall neatly into these classes or have not yet been studied in relation to other genes.

eat genes form a class because they all affect the function of the pharynx, the worm's feeding organ. As mutations in these genes impair food intake and result in the expected developmental and physiological changes, it has been concluded that they prolong life span by causing caloric restriction. Caloric restriction prolongs life span in virtually all animals in which it has been studied, but it remains unclear by which mechanism.

clk genes (clk-1, -2, -3 and gro-1) form a class because mutations in these genes result in the same overall phenotype: in addition to aging, they affect the rates of many physiological processes, including the cell cycle, embryonic and post-embryonic development, behavioral rhythms and reproduction (Lakowski and Hekimi, 1996, Science 272:1010; Wong et al., 1995, Genetics 139:1247). Furthermore, all clk mutants can be maternally rescued, that is, the phenotype of a homozygous clk mutant is wild-type when originating from a heterozygous mother. In spite of the similarities in the phenotypes of clk mutants, the underlying cellular processes that are affected appear to be different for each mutant. Indeed, when clk mutations are combined to create double mutant animals, the effects of the mutations are additive or synergistic for all phenotypes, including aging (Lakowski and Hekimi, 1996, Science 272:1010). Three clk genes have been cloned: clk-1 encodes a mitochondrial hydroxylase that is necessary for ubiquinone biosynthesis (Miyadera et al., 2001, J Biol Chem 276:7713), clk-2 encodes a protein that affects telomere length in worms as well as in yeast, and gro-1 encodes a highly conserved cellular enzyme that modifies a subset of tRNAS.

Mutations in genes that affect the dauer formation pathway form a class for two reasons. Firstly, they all act on the same biological process, the formation of an alternative, dormant, and stress resistant larval stage (the dauer stage) (Riddle, 1988, W. B. Woods Editions, pp. 393-412; Riddle, 1997, Edition Cold Spring Harbor Laboratory Press, pp. 393-412). Secondly, in their action on life span, they all converge on the same intracellular signaling cascade, which involves an insulin receptor-like transmembrane tyrosine kinase (DAF-2) (Kimura et al., 1997, Science 277:942), and ends with a forkhead-like transcription factor (DAF-16) (Ogg et al., 1997, Nature 389:994). The cascade can be said to end with DAF-16 because no mutation known to affect life span and acting genetically downstream of daf-16 has yet been identified, and because the activity of DAF-16 is necessary for the life span prolonging effects of mutations in all other genes in the dauer formation pathway. However, it remains unclear whether the action of daf-2 on daf-16 is the only route by which mutations in daf-2 increase life span.

Much work suggests that the long life of some of the mutants that affect the dauer formation pathway is due to increased stress resistance. In particular, daf-2 mutants are resistant to ROS generating agents, have elevated expression of sod-3 (Honda and Honda, 1999, Faseb J 13:1385), a mitochondrial manganese superoxide dismutase, and their increased life span is abolished by a mutation that decreases the activity of a cytosolic catalase (Taub et al., 1999, Nature 399:162). These observations are highly suggestive, given the large body of data that suggests an involvement of oxidative stress in aging and aging-related diseases. However, there are many other differences between daf-2 mutants and wild-type animals besides resistance to oxidative stress. Thus, the evidence is extensive but only correlative that the long life of daf-2 mutants is in fact due to their greater resistance to oxidative stress.

In Drosophila, it has been possible to increase life span by transgenic expression of enzymes such as SOD and catalase, which protect from oxygen radicals. However, it is difficult to formally demonstrate by these transgenic methods that oxidative stress normally limits the life span of the organism. Indeed, transgene expression might alter the animal's physiology in unpredictable ways that could affect life span only indirectly.

In mice, it has been found that the heterozygous state of sod2(+/−) knockout mice is sufficient to accelerate the aging process in these animals. Also, mutations that abolish the activity of the SHC proto-oncogene locus increase the resistance to ROS of both cultured cells and the whole animal, and also increase life span. How this increased resistance is produced is unclear. One possibility is that the mutant cells have become more resistant to programmed cell death in response to oxidative stress. However, the increased life span of p66^{shc} mutants animals has to date been seen only in the pure inbred 129 strain, which is short lived. It is possible, therefore, that the p66^{shc} mutations only relieves a pathological feature of this strain.

It would be highly desirable to be provided with a new mechanism for altering the aging process or various disease states such as, but not limited to, reactive oxygen species (ROS) mediated diseases, diabetes, hypoxia/reoxygenation injury and Parkinson's disease.

It would also be highly desirable to be provided with a method to identify compounds that can alter the aging mechanism process or be useful in such disease states.

### SUMMARY OF THE INVENTION

One object of the present invention is to provide a detailed molecular, phenotypic and physiological characterization of mutants in the genes isp-1 and ctb-1, whose protein products are involved in electron transport and production of reactive oxygen species in the mitochondrial respiratory chain.

Another object of the present invention is to provide a method to identify compounds that can alter the characteristics of multicellular organisms at the level of cellular physiology, mitochondrial respiration and electron transport, production of reactive oxygen products and resistance to oxidative stress, and developmental, behavioral, reproductive and aging rates.

In accordance with the present invention there is provided an isp-1 gene for use in altering a function at the level of cellular physiology involved in developmental rates, behavioral rates, and longevity, wherein isp-1 mutations cause a longer life and altered developmental and behavioral rates relative to the wild type.

Still in accordance with the present invention, there is provided an isp-1 gene for use altering a function at the level of reactive oxygen species production, wherein isp-1 mutations cause a lower production of reactive oxygen species relative to the wild type.

The isp-1 gene preferably has a sequence as set forth in SEQ ID NO:9, SEQ ID NO:10, SEQ ID NO:11 or SEQ ID NO:12, or an homologue thereof, wherein said homologue codes for a protein having a sequence as set forth in SEQ ID NO:1, SEQ ID NO:2, SEQ ID NO:3, SEQ ID NO:4, SEQ ID NO:5, SEQ ID NO:6, SEQ ID NO:7 or SEQ ID NO:8 or a functional analog thereof, and still preferably said isp-1 gene has a sequence as set forth in SEQ ID NO:12 or codes for a protein having a sequence as set forth in SEQ ID NO:2.

Further in accordance with the present invention, there is provided an ISP-1 protein for use in altering a function at the level of cellular physiology involved in the regulation of developmental rates, behavioral rates, and longevity, wherein said ISP-1 protein is encoded by the gene of the present invention.

In accordance with the present invention, there is also provided an ISP-1 protein for use in altering a function at the level of reactive oxygen species production, wherein said ISP-1 protein is encoded by the gene isp-1 of the present invention.

The present invention also provides for the use of an isp-1 gene to alter a function at the level of cellular physiology involved in the regulation of developmental rates, behavioral rates, and longevity in multicellular organisms, wherein isp-1 mutations cause a longer life and altered physiological rates relative to wild type.

In accordance with the present invention, there is also provided the use of an isp-1 gene to alter a function at the level of reactive oxygen species production in multicellular organisms, wherein isp-1 mutations cause a lower production of reactive oxygen species relative to wild type.

The present invention also provides for the use of the a ISP-1 protein described herein.

Further in accordance with the present invention, there is provided a ctb-1 gene for use in altering a function at the level of cellular physiology involved in developmental rates, behavioral rates, and longevity, wherein ctb-1 mutations cause altered developmental and behavioral rates relative to the wild type.

Still in accordance with the present invention, there is provided a ctb-1 gene for use in altering a function at the level of reactive oxygen species production.

Preferably, the ctb-1 gene has a sequence as set forth in SEQ ID NO:20 or SEQ ID NO:21, or an homologue thereof, wherein said homologue codes for a protein having sequence as set forth in SEQ ID NO:13, SEQ ID NO:14, SEQ ID NO:15, SEQ ID NO:16, SEQ ID NO:17, SEQ ID NO:18 or SEQ ID NO:19, or a functional analog thereof, and still preferably, said ctb-1 gene has a sequence as set forth in SEQ ID NO:21 or codes for a protein having a sequence as set forth in SEQ ID NO:14.

Also in accordance with the present invention, there is provided a CTB-1 protein which has a function at the level of cellular physiology involved in the regulation of developmental rates, behavioral rates, and longevity, wherein said CTB-1 protein is encoded by the gene described above.

In accordance with the present invention, there is provided a CTB-1 protein for use in altering a function at the level of reactive oxygen species production, wherein said CTB-1 protein is encoded by the gene described above.

Further in accordance with the present invention, there is provided the use of the aforementioned ctb-1 gene and CTB-1 protein.

Also in accordance with the present invention, there is provided a method for screening a compound increasing developmental rates, behavioral rates, mitochondrial function, mitochondrial complex III function and/or increasing reactive oxygen species production, comprising the steps of:

a) administering a compound to be screened to an isp-1 mutant organism, or isp-1;ctb-1 double mutant organism; and

b) measuring developmental rates, behavioral rates, mitochondrial function, mitochondrial complex III function and/or production of reactive oxygen species of said mutant of step a),

wherein an increase in developmental rates, behavioral rates, mitochondrial function, mitochondrial complex III function and/or an increased production of reactive oxygen species of said mutant organism, with respect to a wild-type organism is indicative of said compound being useful to manipulate cellular physiology and for therapeutic use in diseases states. Further in accordance with the present invention, there is also provided a method for screening a compound decreasing developmental rates, behavioral rates, mitochondrial function, mitochondrial complex III function and/or decreasing reactive oxygen species production, comprising the steps of:

a) administering a compound to be screened to an organism, and

b) measuring developmental rates, behavioral rates, mitochondrial function, mitochondrial complex III function and/or production of reactive oxygen species of said organism of step a),

wherein a decrease in developmental rates, behavioral rates, mitochondrial function, mitochondrial complex III function and/or a decreased production of reactive oxygen species of said organism, with respect to control organism is indicative of said compound being useful to manipulate cellular physiology and increase longevity, and for therapeutic use in disease states, such as, but not limited to (ROS) mediated diseases, diabetes, hypoxia/reoxygenation injury and Parkinson's disease.

In accordance with the present invention, there is also prodvided a method for screening a compound decreasing or increasing the function of ISP-1 or CTB-1 comprising the steps of:

a) applying a compound to be screened to an in vitro preparation containing ISP-1 or CTB-1, and

b) measuring ISP-1 or CTB-1 activity of said in vitro preparation of step a), wherein an increase or decrease of activity with respect to an untreated preparation is indicative of said coumpound being useful to manipulate cellular physiology, developmental rates, behavioral rates, reactive oxygen species production, longevity and for therapeutic use in disease states, such as, but not limited to (ROS) mediated diseases, diabetes, hypoxia/reoxygenation injury and Parkinson's disease.

In accordance with the present invention, there is further provided a method to increase the life span of a multicellular organism, which comprises decreasing the activity of the isp-1 and/or ctb-1 genes.

Further in accordance with the present invention, there is provided the use of a compound for the manufacture of a medicament for increasing or decreasing physiological rate of tissues, organs, or whole organisms, wherein said compound is altering the activity of the genes isp-1 and/or ctb-1, and/or of ISP-1 and/or CTB-1, in a way that mimics the functional changes produced by the isp-1(qm150) and/or ctb-1(qm189) mutations.

Still in accordance with the present invention, there is also provided the use of a compound for the manufacture of a medicament for increasing or decreasing physiological rate of tissues, organs, or whole organisms, wherein said compound is altering the activity of the genes isp-1 and/or ctb-1, and/or of ISP-1 and/or CTB-1 proteins.

Still in accordance with the present invention, there is further provided a method for screening a compound increasing the function of complex III, isp-1, and/or replacing the function of ubiquinone in complex III, comprising the steps of:

a) administrating a compound to be screened to an clk-1;isp-1 double mutant organism; and

b) assessing viability of said mutant organism of step a), wherein a viable mutant organism is indicative of said compound being useful for manipulating mitochondrial and cellular physiology and for therapeutic use in disease states.

For the purpose of the present invention the following terms are defined below.

The phenotype of an isp-1 mutant is defined as the features of the organisms that are altered in comparison to the wild-type when the protein sequence of the product of the isp-1 gene is altered. Specifically but not exclusively, an isp-1 mutant has slow developmental, behavioral and reproductive rates, as well as low oxygen consumption, high resistance to oxidative stress, a long mean and maximum life span and showing developmental arrest in combination with mutations in clk-1.

The phenotype of an isp-1;ctb-1 mutant is defined as the features of the organisms that are altered in comparison to the wild-type when the protein sequences of the products of the isp-1 gene and of the ctb-1 gene are altered. Specifically but not exclusively, an isp-1;ctb-1 mutant shows feature alterations that are similar to those of isp-1 mutants, but less severe, including for development, behavior and oxygen consumption.

The phenotype of an isp-1;clk-1 mutant is defined as the features of the organisms that are altered in comparison to the wild-type when the protein sequences of the products of the isp-1 gene and the clk-1 genes are altered. Specifcally but not exclusively, a isp-1;clk-1 mutants die as the result of developmental arrest.

### BRIEF DESCRIPTION OF THE DRAWINGS

FIGS. 1A and 1B illustrate the embryonic development and life span of isp-1(qm150) and isp-1(qm150);ctb-1(qm189);

FIGS. 2A to 2C illustrate the low oxygen consumption correlated with high resistance to oxidative stress in isp-1 mutants;

FIG. 3 illustrates the life spans of wild type, isp-1, daf-2, and daf-16 single and double mutants at 20° C.;

FIG. 4 shows the Caenorhabditis elegans protein sequence of Rieske Iron Sulfur Protein (ISP) (SEQ ID NO:1);

FIG. 5 shows the Caenorhabditis elegans protein sequence of ISP in the qm150 mutants (P225S mutation) (SEQ ID NO:2);

FIG. 6 shows the Arabidopsis thaliana protein sequence of ISP (SEQ ID NO:3);

FIG. 7 shows the Gallus gallus protein sequence of ISP (SEQ ID NO:4);

FIG. 8 shows the Saccharomyces cerevisiae protein sequence of ISP (SEQ ID NO:5);

FIG. 9 shows the Bos taurus protein sequence of ISP (SEQ ID NO:6);

FIG. 10 shows the Homo sapiens protein sequence of ISP (SEQ ID NO:7);

FIG. 11 shows the Rattus norvegicus protein sequence of ISP (SEQ ID NO:8);

FIG. 12 shows the Caenorhabditis elegans genomic sequence of the gene isp-1 (F42G8.12) (SEQ ID NO:9);

FIG. 13 shows the Caenorhabditis elegans genomic sequence of the gene isp-1 (F42G8.12) with the qm150 mutation (SEQ ID NO:10);

FIG. 14 shows the Caenorhabditis elegans cDNA sequence for the gene isp-1 (F42G8.12) (SEQ ID NO:11);

FIG. 15 shows the Caenorhabditis elegans cDNA sequence for the gene isp-1 (F42G8.12) with C673T (qm150 mutation) (SEQ ID NO:12);

FIG. 16 shows the ISP-1 protein sequence alignment of Caenorhabditis elegans and its homologues;

FIG. 17 shows the Caenorhabditis elegans protein sequence of Cytochrome b (CTB) (SEQ ID NO:13);

FIG. 18 shows the Caenorhabditis elegans protein sequence of CTB with A170V (qm189 mutation) (SEQ ID NO:14);

FIG. 19 shows the Homo sapiens protein sequence of CTB (SEQ ID NO:15);

FIG. 20 shows the Arabidopsis thaliana protein sequence of CTB (cytochrome b6) (SEQ ID NO:16);

FIG. 21 shows the Bos taurus protein sequence of CTB (SEQ ID NO:17);

FIG. 22 shows the Saccharomyces cerevisiae protein sequence of CTB (SEQ ID NO:18);

FIG. 23 shows the Gallus gallus protein sequence of CTB (SEQ ID NO:19);

FIG. 24 shows the Caenorhabditis elegans sequence for the gene ctb-1 (SEQ ID NO:20);

FIG. 25 shows the Caenorhabditis elegans sequence for the gene ctb-1 with C509T (qm189 mutation) (SEQ ID NO:21); and

FIG. 26 shows the CTB-1 protein sequence alignment of Caenorhabditis elegans and its homologues.

### DETAILED DESCRIPTION OF THE PREFERRED EMBODIMENT

The present invention provides genetic, molecular and physiological traits in Caenorhabditis elegans, which are characteristic of a mutation in the iron sulphur protein of mitochondrial complex III decreasing mitochondrial respiration, resulting in increased resistance to ROS, and increased life span. Furthermore, combining this mutation with a daf-2 mutation that confers protection from ROS does not result in any further increase in life span. Therefore it can be observed that the life span increase observed in the slowly respiring mutant isp-1(qm150) is indeed due to low endogenous ROS. These findings also indicate that the maximum life span increase that can be obtained by decreasing oxidative stress is reached in these mutants.

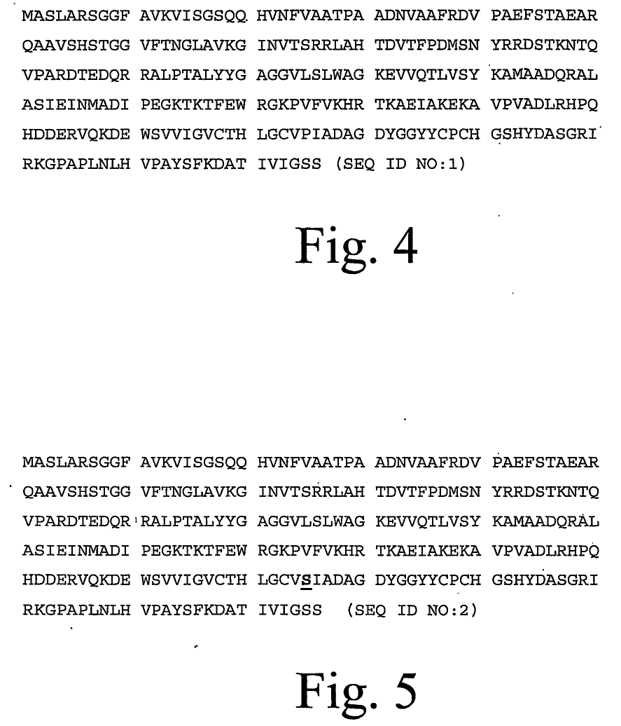

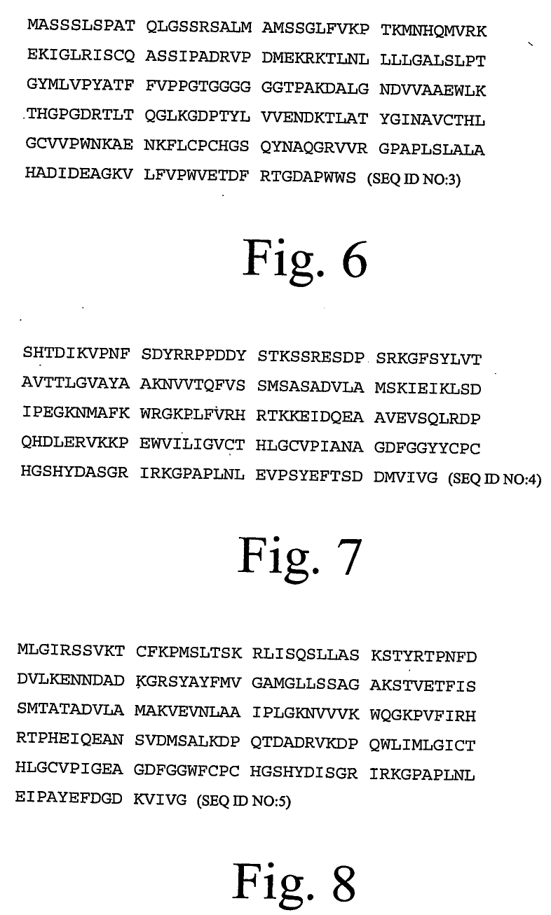

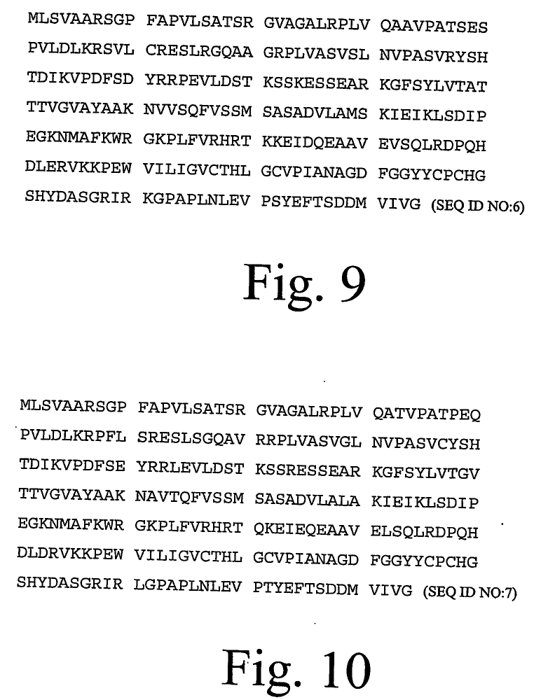

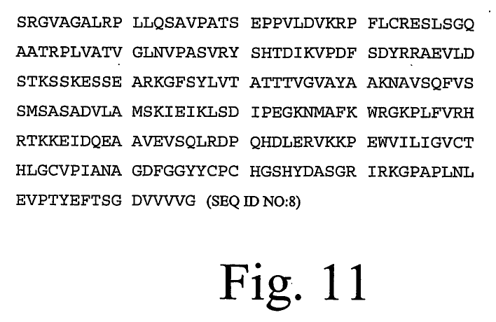

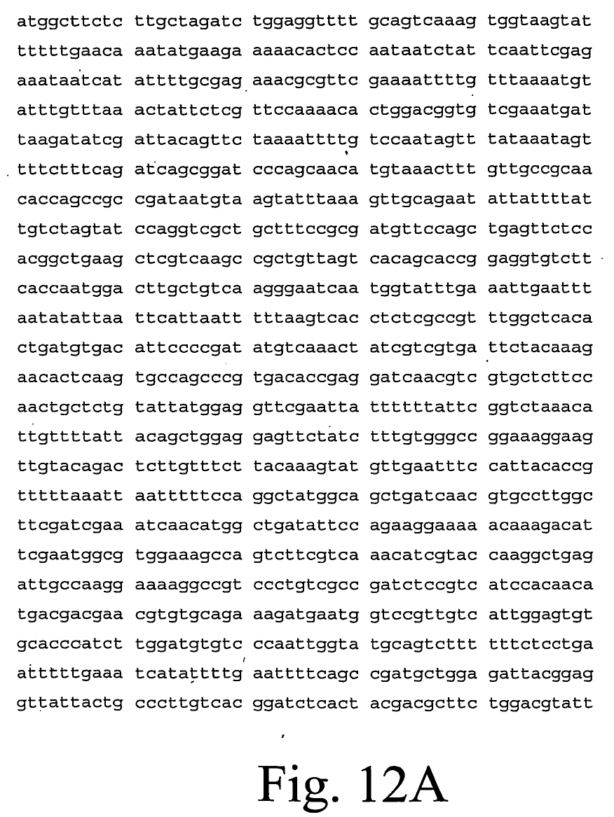

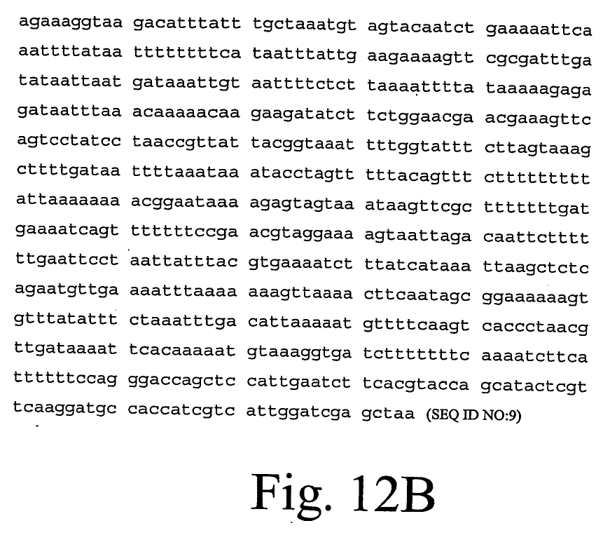

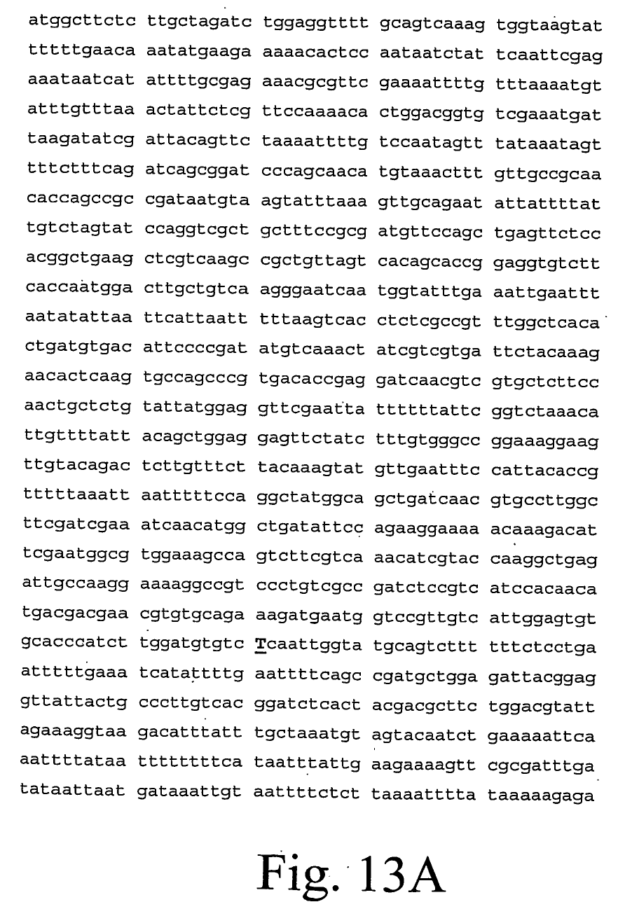

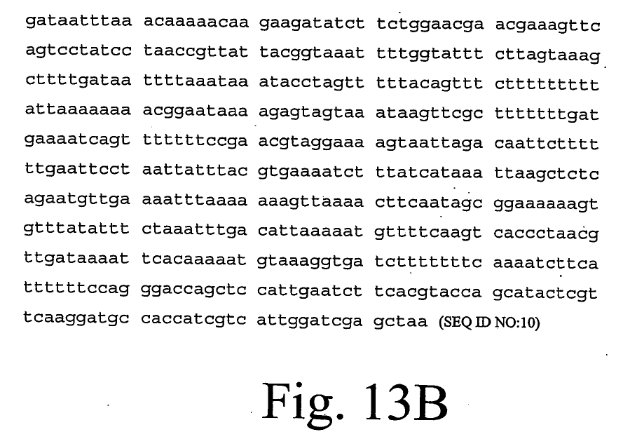

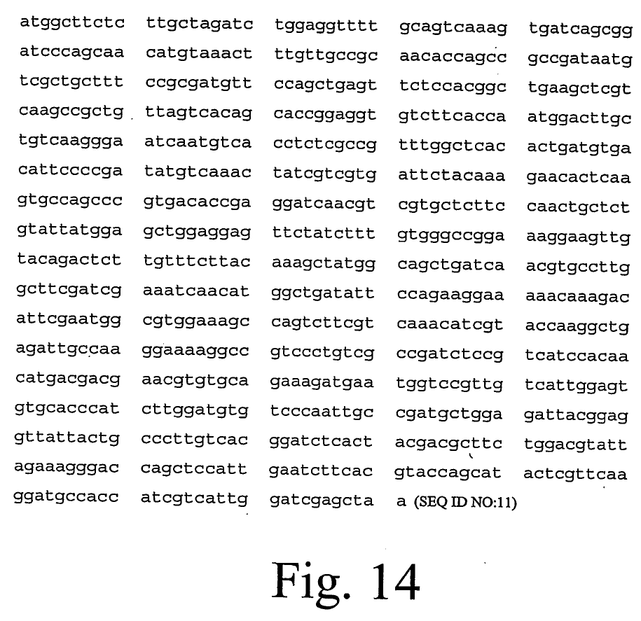

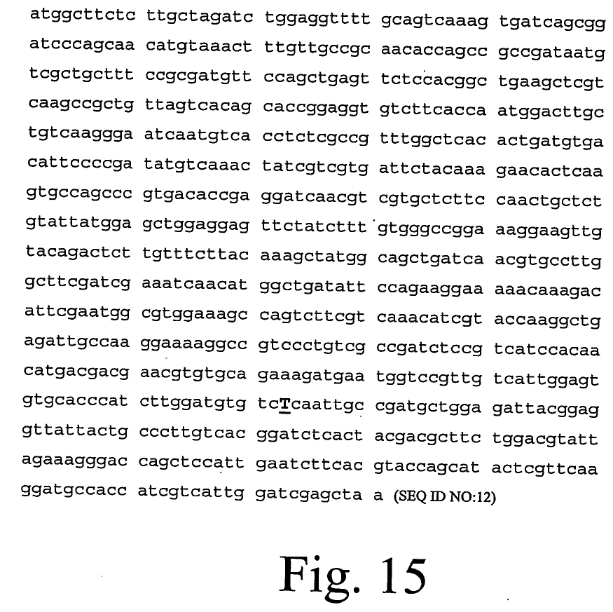

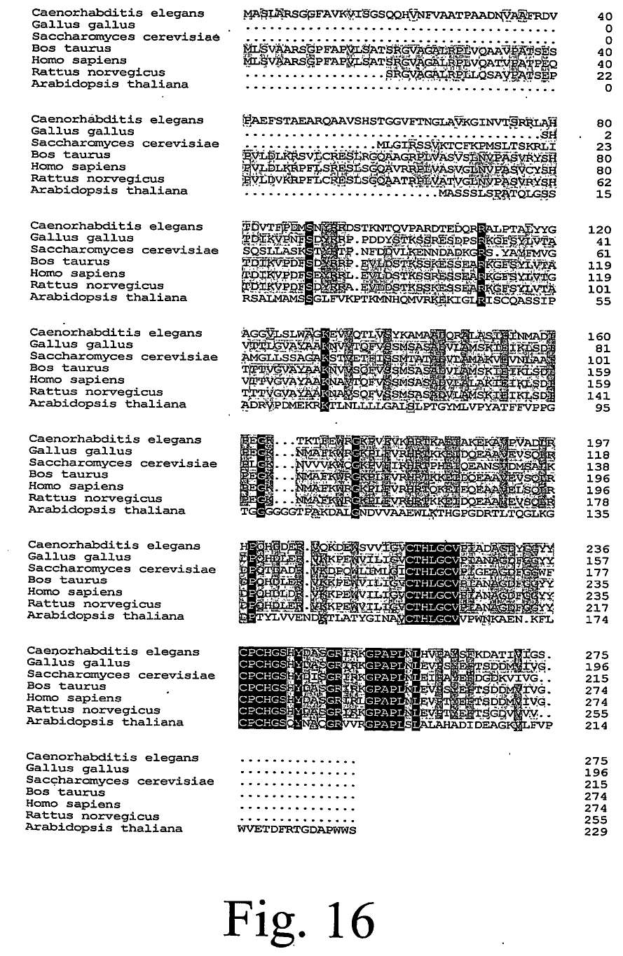

The nucleic sequence of isp-1, its respective protein ISP-1 from different species and mutants according to embodiments of the present invention are described in FIGS. 4 to 15.

A Genetic Screen for Clk-Like Mutants

The major endogenous source of ROS is mitochondrial electron transport. A mutation that reduces electron transport will produce a hypometabolic phenotype not very different from that of severe clk mutants, that is, slowed physiological rates, in particular, slowed development, behaviors and reproduction.

In order to identify electron transport chain mutations, a genetic screen for mutants that grew slowly and had an increased defecation cycle length was carried out. The screen was carried out on animals of the F2 generation after mutagenesis. Wild-type animals were mutagenized with ethyl methane sulfonate (EMS) following the standard protocol. Animals from the F2 generation that grew very slowly and had a slow defecation cycle were plated individually. Those animals that produced an entire brood of slow developing mutant worms without physical abnormalities were analyzed further. All phenotypic assays including the measures of development were carried out as in Wong et al., (1995, Genetics 139:1247), and life span was measured as in Lakowski and Hekimi (1996, Science 272:1010). A number of mutants were identified in this screen. The detailed analysis of one of the mutants (qm150) is presented here (FIG. 5).

qm150: a Healthy Mutant With Very Slow Physiological Rates

TABLE 1 shows a quantitative analysis of the main developmental, behavioral and reproductive features of the mutant. All timed physiological rates, including aging, are much slower (1.5 to 5-fold) in the mutant than in the wild type, with the egg-laying rate being the most severely affected feature. Remarkably, in spite of these dramatic phenotypes, these animals appear very healthy with, for example, virtually no increase in embryonic or post-embryonic lethality in comparison to the wild type (TABLE 1).

TABLE 1
Phenotype of isp-1 and isp-1; ctb-1 mutants at 20° C.

| | Wild type(N2) | | |
|---|---|---|---|
| | Mean ± s.d. | | isp-1(qm150); |
| Genotype | (sample size) | isp-1(qm150) | ctb-1(qm189) |
| Embryonic Lethality | 0.7% | 1.9% | 1% |
| | (n = 300) | (n = 310) | (n = 300) |
| Post-embryonic | 1% | 2.5% | 0.5% |
| lethality | (n = 300) | (n = 310) | (n = 300) |
| Embryonic | 15.3 ± 1.3 | 25.7 ± 2.4 | 15.1 ± 1.1 |
| Development (hours) | (n = 103) | (n = 124) | (n = 45) |
| Post-embryonic | 44.9 ± 1.6 | 92.8 ± 2.4 | 68.3 ± 2.3 |
| development (hours) | (n = 120) | (n = 96) | (n = 262) |
| Defecation cycle | 57.3 ± 1.8 | 121.3 ± 2.1 | 89.5 ± 12.1 |
| (seconds) | (n = 25) | (n = 25) | (n = 25) |
| Egg Production Rate | 6.4 ± 0.5 | 1.3 ± 0.4 | 2.8 ± 0.2 |
| (eggs/hour) | (n = 100) | (n = 100) | (n = 100) |
| Self-brood size | 319.7 ± 21.8 | 82.5 ± 6.4 | 175.7 ± 10.3 |
| | (n = 20) | (n = 20) | (n = 20) |
| Oxygen consumption | 32.1 ± 8.9 | 13.8 ± 3.7 | 18.6 ± 7.3 |
| (nmoles O₂/min/mg | (n = 5) | (n = 4) | (n = 5) |
| protein ± s.e.m) | [1.8] | [4.8] | [4.1] |
| [Cyanide-resistant | | | |
| oxygen consumption] | | | |

qm189: a Spontaneous Mitochondrial Suppressor of qm150

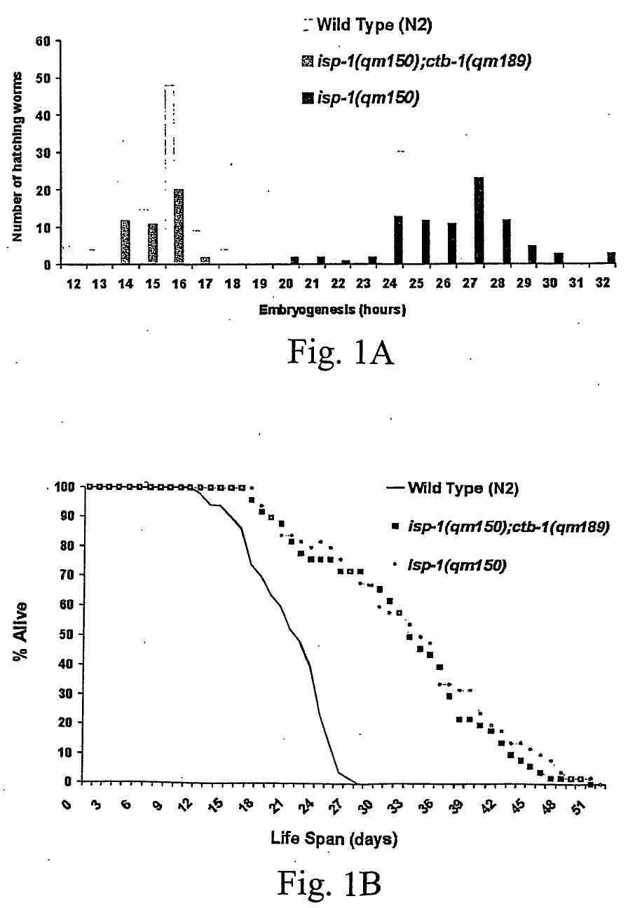

During the process of culturing the qm150 strain, a rare spontaneous partial suppressor of qm150 phenotype was identified. This suppressor completely suppresses the slow embryonic development of qm150 mutants (FIG. 1 and TABLE 1), and partially suppresses the slow post-embryonic development as well as the slow behavioral and reproductive features (TABLE 1), but does not change the very long life span of qm150 (FIG. 1 and TABLE 2).

TABLE 2
Life spans of mutants at 20 and 25° C.

| | | | | | | isp-1(qm150); | daf-16(m26); | daf-2(e1370); |
|---|---|---|---|---|---|---|---|---|
| | | Wild Type | daf-2(e1370) | daf-16(m26) | isp-1(qm150) | ctb-1(qm189) | isp-1(qm150) | isp-1(qm150) |
| 20° C. | Mean Life | 19.6 ± 4.7 | 36.3 ± 12.3 | 17.0 ± 3.4 | 33.0 ± 9.7 | 32.8 ± 9.4 | 29.6 ± 7.3 | 42.3 ± 14.8 |
| | Span ± s.d. (days) | | | | | | | |
| | Adult Life Span* | 17.6 | 33.3 | 15 | 28 | 29.8 | 23.6 | 33.3 |
| | Maximum | 31 | 66 | 28 | 63 | 52 | 54 | 108 |
| | Life Span | | | | | | | |
| | Sample Size | n = 250 | n = 250 | n = 300 | n = 283 | n = 100 | n = 325 | n = 227 |
| 25° C. | Mean Life | 12.4 ± 3.5 | 32.9 ± 12.0 | n.d. | 22.0 ± 7.8 | 18.7 ± 6.0 | n.d. | 41.1 ± 13.3 |
| | Span ± s.d. (days | | | | | | | |
| | Adult Life Span* | 10.4 | 29.9 | n.d. | 16 | 13 | n.d. | 32.1 |
| | Maximum | 21 | 56 | n.d. | 39 | 31 | n.d. | 71 |
| | Life Span | | | | | | | |
| | Sample Size | n = 50 | n = 50 | n.d. | n = 50 | n = 50 | n.d. | n = 50 |

*The adult life span is obtained by substracting the known developmental time in days of each strain from the total life span.
n.d.: Not determined.

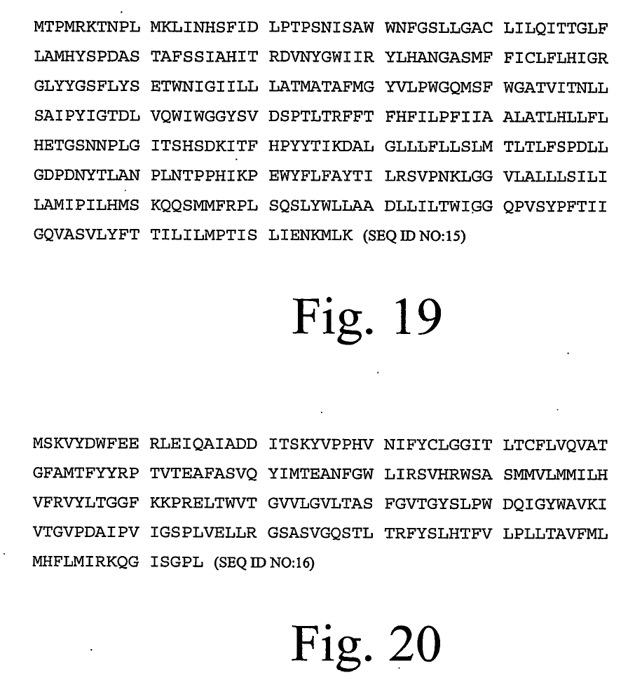

In the process of analyzing this suppressor genetically it was discovered that it displayed cytoplasmic inheritance and, thus, was likely to be encoded by the mitochondrial genome. In brief, animals of the strain carrying both the qm150 and the suppressor (qm189) (FIG. 18) mutations were crossed to wild-type males and their F1 hermaphrodite progeny was allowed to self-fertilize. The resulting F2 progeny was analyzed and it was found that all slowly developing animals (homozygous for qm150) were still suppressed, and thus appeared to be still carrying qm189. In the reciprocal cross, however, when the double qm150 qm189 mutant males were mated to wild-type hermaphrodites, and the F2 progeny was analyzed, none of the slowly developing animals (qm150 homozygotes) were suppressed, that is, they were all developing as slowly as animals of the original qm150 strain, and therefore did not carry the qm189 mutation. These results are consistent with the suppressor mutation segregating with the cytoplasm of oocytes and therefore being mitochondrial.

qm150 is a Mutation in the Iron Sulfur Protein of Complex III of the Mitochondrial Electron Transport Chain

qm150 on chromosome IV was mapped, immediately to the right of unc-24 (0.06-0.11 cM of unc-24). The gene was molecularly identified by transformation rescue using genomic clones and PCR amplification products from that region. Several cosmids covering the predicted region were microinjected in isp-1(qm150) worms. The cosmid F42G8 was able to rescue the isp-1 phenotypes. A genomic region containing only the predicted gene F42G8.12, which codes for the worm iron sulfur protein (ISP) of mitochondrial complex III, was PCR amplified from the cosmid with primers SHP1587 (GCTCCGCCTCATCTAGAGAACCTC) and SHP1588 (CAGTCCGCCCGTTGAGTTTGTCCC) and was sufficient for full phenotypic rescue. A 2.4 kb fragment encompassing the whole gene was injected in worms and found to rescue the slow development and slow defecation of twp-1(qm150) animals.

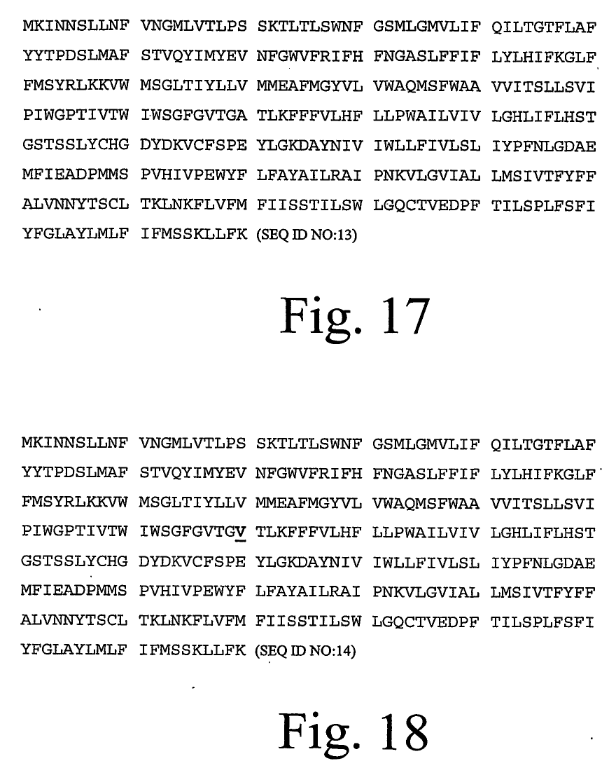

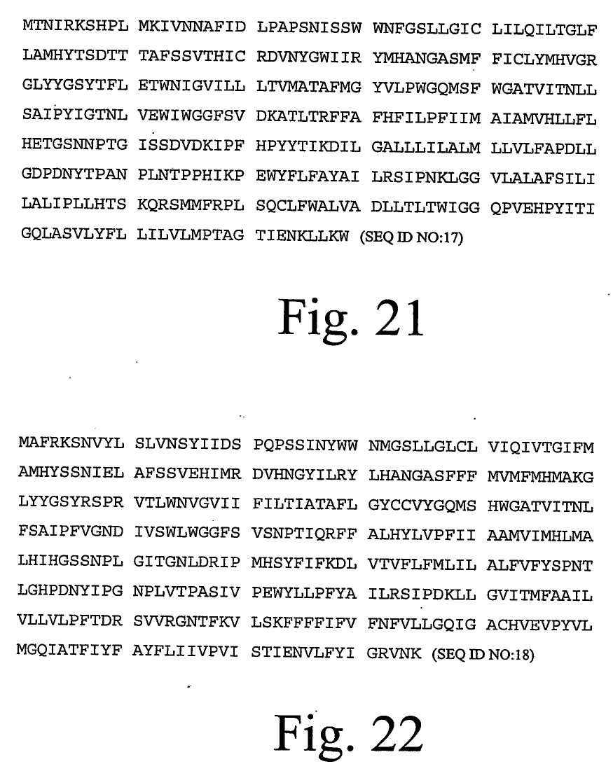

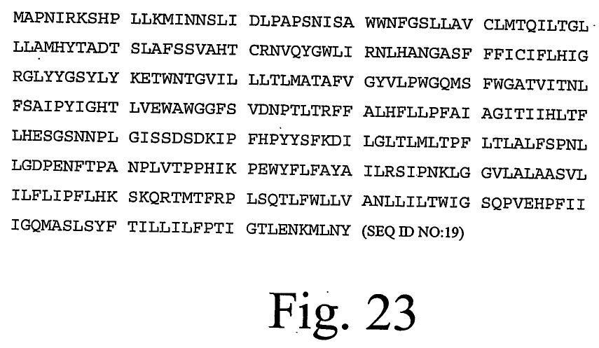

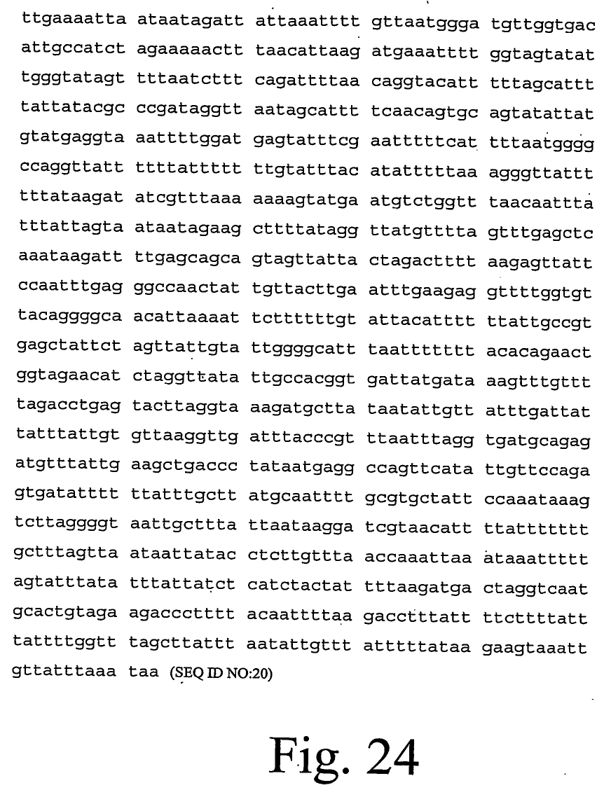

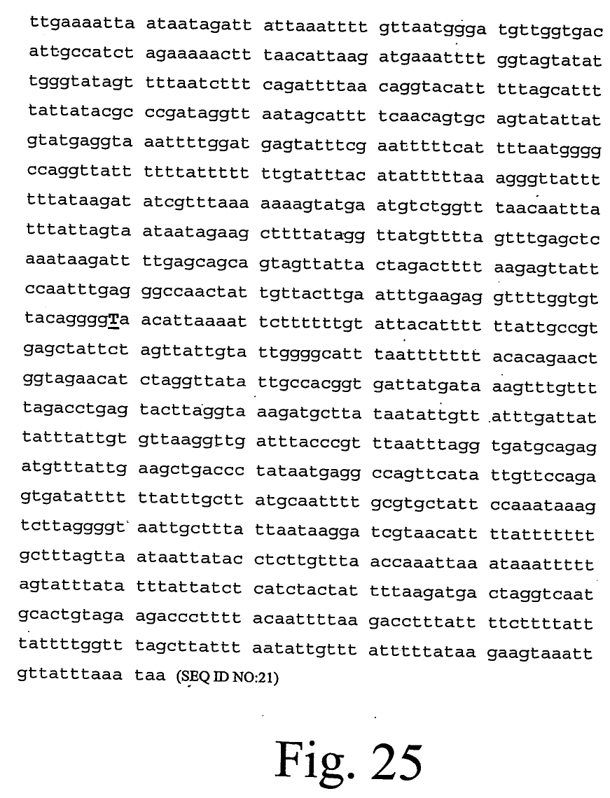

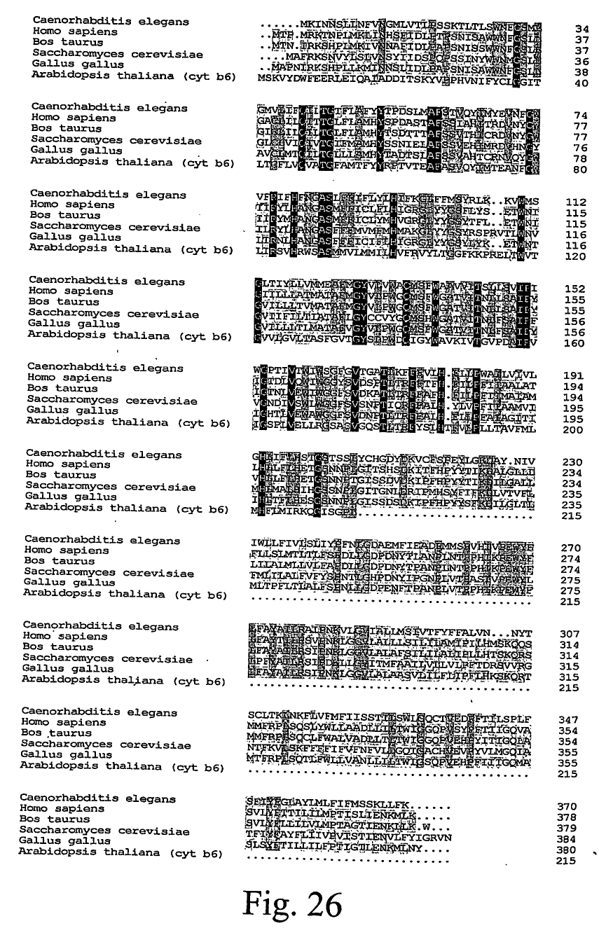

Mitochondrial complex III catalyzes electron transfer from ubiquinol to cytochrome c. FIGS. 17 to 26 show the nucleic sequences of ctb-1 with different mutants and their respective proteins CTB-1. It is composed of three subunits that catalyze the redox reactions that are carried out by the complex (cytochrome b, the iron sulfur protein, and cytochrome c1) and of a number of additional subunits. The three catalytic subunits are highly conserved in all mitochondria and aerobic bacteria. The ISP carries a 2Fe-2S prosthetic group that is held in place by two histidine and two cysteine residues. The F42G8.12 gene was PCR amplified with primers SHP1587 and SHP1588 from twp-1(qm150) worms. By comparing with published sequences a C to T transition was found at position 673 of the F42G8.12 gene. This mutation was not observed when sequencing this gene region from N2 wild-type strain. Thus, isp-1(qm150) is a point mutation at residue 225 that changes a proline into a serine. Prolines are known to be important structurally, as they make the peptide backbone locally rigid. Furthermore, proline 225 is in close proximity to the prosthetic group and is part of the structure that holds it in place. Therefore the mutation directly affects the properties of the iron sulfur centre.

qm189 is a Mutation in the Mitochondrially Encoded Cytochrome b of Complex III

Cytochrome b is the only subunit of complex III that is encoded by the mitochondrial genome. PCR-amplification was performed and the cytochrome b locus (ctb-1) from the mitochondrial DNA of the suppressed strains carrying qm189 was sequenced. Two PCR fragments (596 bp and 764 bp) encompassing the whole cytochrome b gene were amplified from isp-1(qm150);cyb-1(qm189) genomic DNA using two overlapping sets of primers (SHP1622 (CCCTGAAGAG GCTAAGAATA TTAGG):SHP1633 (CAATACAATA ACTAGAATAG CTCACGGC) and SHP1623 (GATCTTAACA TTCCGGCTGA GGC):SHP1632 (GGTTTTGGTG TTACAGGGGC)). The amplicons were then sequenced in both directions. The cytochrome b gene was found to contain a C to T transition at position 509, leading to an alanine to valine change at residue 170. The presence of a unique peak on the sequencing chromatograms suggests that the mutation is homoplasmic. This mutation was not present in N2 and isp-1(qm150) worms and other wild-type isolates. The mutation appears to be homoplasmic, that is, all the mtDNA molecules carry the mutation, as no signal corresponding to the wild-type sequence was observed in the product amplified from the ctb-1(qm189) strain.

Furthermore, the mutation cannot be lost from the mitochondrial DNA pool, even in the absence of phenotypic selection by the presence of the isp-1(qm150) mutation. Indeed, when the isp-1(qm150);ctb-1(qm189) strain is backcrossed with wild-type males and the suppressor mitochondria are kept associated with a wild-type nuclear genome for a few generations, all mtDNA still carries A170V, and the mutant cytoplasm is still capable of suppressing the isp-1(qm150) phenotype when in a qm150 homozygous background. Indeed, unc-24(e138) heterozygous males were crossed with isp-1(qm150);ctb-1(qm189) hermaphrodites and a F2 homozygous unc-24(e138) line was established. Such a line necessarily harbors ctb-1 (qm189). unc-24(e138);ctb-1(qm189) hermaphrodites from this line were crossed with isp-1(qm150) males and homozygous isp-1(qm150);ctb-1(qm189) were isolated. When scoring defecation cycles and growth rate it appeared that the ctb-1 cytoplasm was still present and able to partially suppress the Isp-1 phenotype. The cytochrome b gene was PCR amplified and sequenced from both the unc-24(e138);ctb-1(qm189) and isp-1(qm150);ctb-1(qm189) mutants. The same homoplasmic C to T transition at position 509 was found in both unc-24(e138);ctb-1(qm189) and isp-1(qm150);ctb-1(qm189) strains suggesting the persistence of the mutation in a non isp-1 genetic background.

These findings are consistent with the mechanism of electron transfer from ubiquinol at its binding site on cytochrome b to cytochrome c1, which involves a molecular movement and a change of conformation of the head of ISP (that carries the 2Fe-2S group) from a docking surface on cytochrome b, where it has received an electron from ubiquinol, to a position where it interacts with cytochrome c1. On its way back from interacting with c1, the ISP head also adopts an intermediate position before fully docking on cytochrome b. This intermediate position serves to ensure that a new molecule of ubiquinol has replaced the ubiquinone from the previous round in the ubiquinone cycle, before the ISP docks at the site for electron transfer from ubiquinol to the 2Fe-2S. Alanine 170 is located immediately adjacent to a highly conserved threonine at the beginning of a loop known to form part of the docking site.

Oxygen Consumption is Reduced in isp-1(qm150) Mutants

The phenotype of isp-1 and isp-1;ctb-1 is consistent with a slow down of the rate of electron transfer (and thus ATP generation) by the isp-1(qm150) mutation, and ctb-1(qm189) partially re-establishes a higher rate. An altered redox potential is responsible for the iron sulfur centre that slows down the rate of electron transfer from ubiquinol, or for a slower rate of conformational change of the ISP head that mediates transfer from cytochrome b to cytochrome c1. The mutation in CTB-1 alters both these parameters when ISP-1 is docked on the CTB-1 protein. Thus, the rate of oxygen consumption of isp-1(qm150) mutants is decreased and is partially re-established by ctb-1(qm189). The oxygen consumption is measured by placing live animals in a closed chamber and monitoring oxygen concentration with an oxygen electrode (see TABLE 1). Worms from 10-15 large NGM plates were bleached to extract eggs. Eggs were allowed to hatch overnight at 20° C. in M9 buffer. L1 larvae were transferred to large seeded NGM plates and fed ˜3 hours at 20° C. The L1s were collected, washed free of bacteria by sucrose flotation, re-suspended in M9 and incubated at 20° C. for 45 min. Oxygen concentration was monitored with a Clark electrode in a closed chamber for ˜10 min. Worms were then collected, pelleted and kept at −80° C. for protein quantification.

The oxygen consumption of the isp-1 mutants is indeed reduced approximately 2-fold, and ctb-1(qm189) indeed partially re-establishes a higher oxygen consumption. It was found that oxygen consumption in the wild type and the mutants is cyanide sensitive, indicating that this consumption is indeed the result of reduction by electrons that have been transported along the respiratory chain.

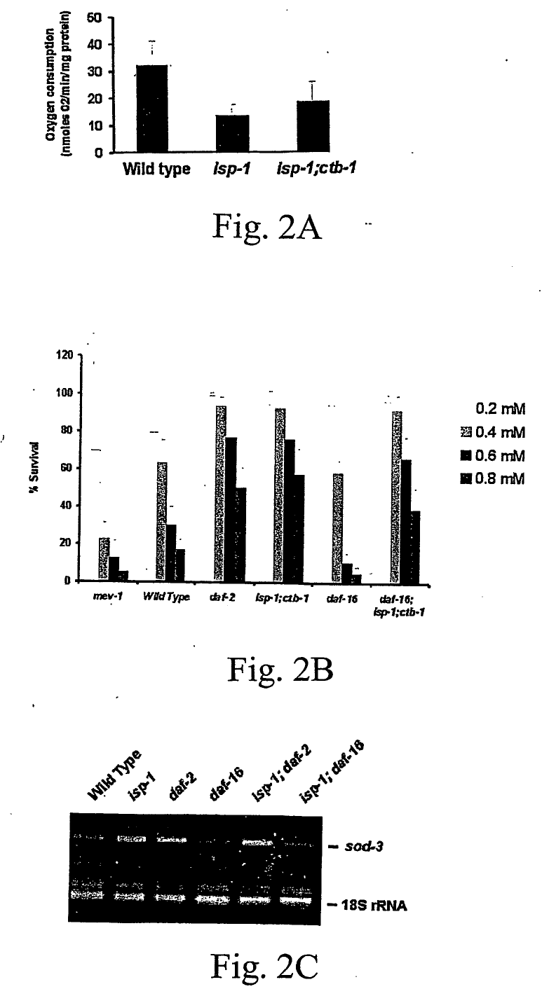

The effect of ctb-1(qm189) on oxygen consumption is not marked compared to its dramatic effect on the phenotype of isp-1(qm150). For example, ctb-1(qm189) re-establishes a wild-type rate of embryonic development to isp-1 mutants, which develop two times more slowly than the wild type (TABLE 1; FIG. 2A). These observations show that embryonic development does not require a very high level of respiration, and also, that the level of respiration in isp-1 mutants is just below a critical threshold below which some physiological functions become severely impaired.

Developmental Arrest of clk-1;isp-1 Double Mutants

The double mutants clk-1(e2519);isp-1(qm150) and clk-1(qm30); isp-1(qm150) were constructed. Both combinations lead to developmental arrest, whether the clk-1 or the isp-1 mutations are made homozygous first. Co-enzyme Q (ubiquinone; CoQ) is a critical co-factor of the function of the bc1 complex of which ISP-1 is one of the three enzymatically active components. Co-enzyme Q is bound on cytochrome b (ctb-1) and an electron is transferred from the bound CoQ to the iron sulphur center of ISP-1. CoQ is entirely absent from clk-1 mutants (Miyadera et al., 2001) and is replaced by demethoxyCoQ (DMQ). DMQ is known to function adequately, but less efficiently, in the respiratory chain. The findings of lethality in the double mutants clk-1(e2519);isp-1(qm150) and clk-1(qm30); isp-1(qm150) indicate that the combination of a functionally altered iron sulphur protein and a functionally altered co-factor results in an overall insufficient function of the complex.

Resistance to Oxidative Stress

One of the main sources of oxidative stress in the organism is the superoxide that is produced when a ubisemiquinone species is generated at complex III. The semiquinone can donate electrons to oxygen and thus produce superoxide. Superoxide, which is highly reactive, can be detoxified into peroxide (H₂O₂) by the enzyme superoxide dismutase (SOD). Peroxide, which is still reactive and can be the source of the highly reactive hydroxyl ion, can be further detoxified by various enzymes, including catalase.

The low oxygen consumption of isp-1(qm150) mutants indicated that their long life span is due to low production of reactive oxygen species (ROS) and, consequently, to a low rate of molecular damage accumulation. The mutants' resistance to oxidative stress produced by an exterior source such as paraquat was examined. When paraquat is taken up by cells, superoxide is produced under the influence of the intracellular redox conditions. Resistance or hypersensitivity to paraquat has been widely used to test for how cells and organisms are able to cope with oxidative stress (e.g. Honda and Honda, 1999, supra). If endogenously produced levels of ROS are low in the mutants, they should be able to cope better with the extra oxidative stress produced by paraquat and become relatively resistant to this compound.

The paraquat resistance was tested by scoring the proportion of animals that succeed in completing development when placed on plates containing various concentrations of paraquat (FIG. 2B). For each strain tested, 100 L1 animals were placed to develop to adulthood on NGM plates containing different concentrations of paraquat (0 mM, 0.2 mM, 0.4 mM, 0.6 mM and 0.8 mM). For each strain, worms were monitored each day until 6 days after the first worm becomes adult. The survival was expressed as the percentage of worms that reached adulthood. Each strain was tested at least three times. Exposure to paraquat lengthens substantially the duration of development of all animals, including the wild type. Therefore, very slowly developing mutant strains, such as those carrying unsuppressed isp-1(qm150), whose development is twice as long as that of the wild type, become exposed to paraquat for such a long time that they never complete development and therefore cannot be tested in this way. This does not mean that they are hypersensitive to oxidative stress, but that the length of their exposure to the toxic effects of paraquat overwhelms any defenses. It was found that isp-1;ctb-1 mutants, whose development is only 40% slower than that of the wild type (TABLE 1), are very resistant to paraquat compared to the wild type. As expected, it was also found daf-2 mutants to be resistant by this test. It is of note that this is not a property of all mutations affecting the respiratory chain in worms. For example, mutants of mev-1, which encodes a subunit of complex II, are hypersensitive to paraquat by this test (FIG. 2B), as are mutants of gas-1, which encodes a subunit of complex I.

Resistance to oxidative stress in the daf-2 system is correlated with high levels of expression of sod-3. To test whether the resistance of isp-1 mutants to oxidative stress is due to a similar mechanism, the level of sod-3 expression was tested by quantitative PCR. Relative quantitative RT-PCR was used to compare the expression of sod-3 mRNA between worm strains. RNA from L1 larvae and young adults was extracted with Trizol™. Reverse transcription were carried out using Superscript™ II RNase H⁻ Reverse Transcriptase (Gibco BRL Life Technologies) with random hexamer primers according to the manufacturer's recommendation. Different amounts of RNA (from 10 pg to 100 ng) from a given sample (e.g. N2 worms) were used for RT-PCR amplifications. The amount of RNA was chosen to give a detectable sod-3 band after 35 cycles of PCR to prepare cDNA for subsequent experiments. PCR amplification was performed using sod-3 specific primers, one tenth of the cDNA preparation and Taq DNA polymerase (Qiagen) with the following regimen: 45 sec at 94° C., 45 sec at 58° C. and 45 sec at 72° C. with prior denaturation at 94° C. for 1 min and a 3 min final extension at 72° C.

To get reliable comparisons between samples (worm strains), the exponential phase of the PCR reaction was determined. For this [\tilde{□}³²P]dCTP was added to the PCR tubes and PCR amplification was run for 39 cycles. Samples were removed every two cycles starting at cycle 17. PCR products were separated by electrophoresis and quantified using a Phospholmager. The signal was then plotted against the cycle number. A cycle number was chosen in the middle of the exponential phase to perform the relative quantitative RT-PCR experiments. For this, cDNA from samples to study were used for non-radioactive PCR amplification using the above conditions except that specific 18S rRNA specific primers were added as an internal control (QuantumRNA™ 18S Internal Standards, Ambion). The system provided by Ambion allows to attenuate the signal from the abundant 18S rRNA to reach the level of the target gene thus allowing more accurate comparison. PCR products were separated on 1.5% agarose gels and stained with SYBR Gold (Molecular Probes).

High levels of sod-3 in isp-1 mutants were observed, similar to those found in daf-2 (FIG. 2C), indicating that the worms react to the impaired function of the respiratory chain produced by isp-1(qm150) by increasing protection from ROS. The increase in sod-3 expression in daf-2 mutants as well as all forms of stress resistance in these mutants, including oxidative stress, are fully suppressed by mutations in daf-16. To test whether the same is true for isp-1 mutants, a daf-16;isp-1;ctb-1 triple mutant strain was constructed and tested it for sod-3 levels and resistance to paraquat. To construct daf-16(m26)1; isp-1(qm150)IV; ctb-1(qm189) triple mutants, an unc-29(e193)1; isp-1(qm150)IV; ctb-1(qm189) strain was first constructed by crossing unc-29(e193) males to isp-1(qm150); ctb-1(qm189) hermaphrodites; then daf-16(m26) males were crossed to unc-29(e193); isp-1(qm150); ctb-1(qm189) hermaphrodites and non-Unc hermaphrodites that produced no Unc progeny were picked to produce the daf-16(m26); isp-1(qm150); ctb-1(qm189) strain. The presence of daf-16(m26) was confirmed by sequencing of the m26 allele.

It was found that daf-16 prevents the increased expression of sod-3, but that the triple mutants are as resistant to paraquat as isp-1;ctb-1 double mutants or daf-2 mutants. These findings indicate that, although isp-1;ctb-1 mutants have low endogenous ROS production as well as increased stress resistance, the increased resistance of daf-16;isp-1;ctb-1 to paraquat is mostly or even entirely due to low endogenous ROS production. These findings also indicate that daf-16 is part of the mechanism that increases protection from ROS in response to impaired mitochondrial respiration.

Long Life Span of daf-16;isp-1

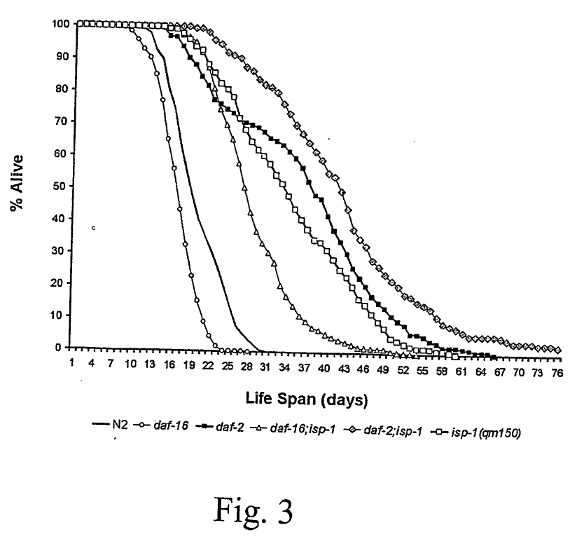

A wild-type daf-16 allele is not only necessary for the stress resistant phenotypes of daf-2 mutations and other mutations in the dauer pathway, but also for their long life. daf-16(m26)I; isp-1(qm150)IV double mutants was constructed by crossing daf-16(m26)I males to unc-29 (e193)I; isp-1(qm150)IV double mutant hermaphrodites. Non-Unc slow developing F2 worms were kept. The presence of daf-16(m26) was confirmed by sequencing of the m26 allele. It was found that daf-16 is not necessary to account for most of the longevity of isp-1(qm150) (FIG. 3 and TABLE 2). This observation shows that isp-1 does not exercise its life prolonging effects by indirectly regulating daf-2. Also, the high level of sod-3 expression in isp-1, which is suppressed by daf-16, is not necessary for the long life of isp-1(qm150). Although the life span of the double mutant is somewhat shorter than the life span of isp-1(qm150), it is still dramatically longer than the life span of daf-16 (FIG. 3 and TABLE 2). The moderate shortening of life span by daf-16 in the double with daf-16;isp-1 double mutants are due to the life span-shortening effects of daf-16 mutations per se (FIG. 3 and TABLE 2), and/or to the fact that the high level of sod-3 and other activities controlled by daf-16, are minor contributors to the longevity of isp-1(qm150).

The Effects of isp-1 and daf-2 on Life Span are not Additive

The molecular nature of ISP-1 and CTB-1, the low respiration of the mutants, and their daf-16-independent resistance to oxidative stress, indicate that isp-1 mutants produce low levels of ROS. However, this does not formally demonstrate by itself that the long life of these mutants is the direct consequence of their low levels of oxidative stress. For example, the long life span could be due to any damaging process whose rate is reduced as a consequence of slow respiration. In this view, the resistance phenotype would be merely a side-effect that is not crucial for life span in the absence of an environmental increase in oxidative stress. To test this, daf-2,isp-1 double mutants were constructed and their life span studied. daf-2(e1370)III, isp-1(qm150)IVdouble mutants were prepared by mating daf-2(e1370)III hermaphrodites to isp-1(qm150)IV males at 20° C. Slow growing F2 animals were picked (isp-1 mutant phenotype) and their F3 progeny were placed as eggs at 25° C. Those animals that arrested as dauers were kept as putative daf-2(e1370); isp-1(qm150) double mutant strains and transferred to 15° C. to recover and finish development. The daf-2; isp-1 double mutants were scored for slow development rates and a dauer constitutive phenotype at 25° C. to confirm the presence of isp-1(qm150) and daf-2, respectively. It was found that the effects of daf-2 and isp-1 on life span are almost identical in magnitude but are not additive as the double mutants live only marginally longer than any one of the single mutants (FIG. 3 and TABLE 2). Furthermore, this slight increase in the mean life span of the double can be entirely attributed to their very slow development (the double mutants take 9 days to develop, which is 4 days more than of isp-1) (TABLE 2). These results show that isp-1 mutants live long because of low endogenous ROS production and that daf-2 mutants live long because of high levels of enzymatic activities that protect from the damaging effects of ROS. In addition, they indicate that the maximal life span increase that can be obtained by reducing oxidative stress is reached in both mutants. Indeed, if the life span of isp-1 mutants is already not limited by oxidative damage thanks to low ROS production, then extra protection from ROS conferred by daf-2 does not improve much on life span (FIG. 3).

The above requires that the daf-2 mutation still exercises its effects in an isp-1 background. Three observations indicate that this is the case. 1) The double mutants are dauer-constitutive at 25° C. 2) The increase in the expression of sod-3 in the double mutants is even larger than in the single mutants (FIG. 2C). 3) It was found that the life spans of isp-1 and the wild type at 25° C. are not as long as at 20° C., because the respiratory chain is strained to meet higher metabolic demand, which results in increased ROS production (TABLE 2). However, the daf-2;isp-1 double mutants live as long as daf-2 mutants and much longer than isp-1 mutants at 25° C. (TABLE 2). This indicates that the daf-2 mutation is still fully capable to exercise its protective and life-prolonging effect in an isp-1 background.

Therefore, it has been observed that a decrease in the function of the respiratory chain can dramatically increase animal life span. The present experiments also demonstrate that this life span increase is due to a lower rate of endogenous ROS production. Indeed, isp-1 mutants are resistant to exogenously imposed oxidative stress and their life span cannot be lengthened through the action of a mutation in daf-2 that increases ROS resistance. At the same time these findings demonstrate that the increased oxidative stress resistance of daf-2 mutants is the source of their increased life span, as a daf-2 mutation has little effect on the longevity of animals (isp-1 mutants) that already have low oxidative stress. Finally, the characteristics of these two mutants and the fact that their effects on life span are not additive indicate that the maximum life span increase that can be obtained by protection from ROS is less than two-fold and is approximately that of daf-2 or isp-1 mutants.

Production of reactive oxygen species by the mitochondrial respiratory chain has been implicated in numerous human diseases, including, but not exclusively, diabetes (Nishikawa et al., 2000, Nature 404:787-790; Du et al., 2000, PNAS 97(22):12222-12226; and Brownlee, 2001, Nature 414:813-820), hypoxia/reoxygenation injury (Lefnesky et al., 2001, J Mol Cell Cardiol., 33 :1065-1089; Cuzzocrea et al., 2001, pharmacol review, 53:135-159) and Parkinson's. Our findings that a specific alteration of the function of ISP-1 can lower the level of ROS production by the mitochondrial respiratory chain, allows for the discovery of drugs and methods that target mitochondrial complex III and ISP-1 in humans to alleviate disease. In particular, one approach is to find compounds and methods that modify the function of ISP-1 in the same way as it is modified by the qm150 mutation.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A isp-1 gene for use in altering a function at the level of cellular physiology involved in developmental rates, behavioral rates, and longevity, wherein isp-1 mutations cause a longer life and altered developmental and behavioral rates relative to the wild type.

2. A isp-1 gene for use altering a function at the level of reactive oxygen species production, wherein isp-1 mutations cause a lower production of reactive oxygen species relative to the wild type.

3. The isp-1 gene of claim 1, wherein said isp-1 gene has a sequence as set forth in SEQ ID NO:9, SEQ ID NO:10, SEQ ID NO:11 or SEQ ID NO:12, or an homologue thereof, wherein said homologue codes for a protein having a sequence as set forth in SEQ ID NO:1, SEQ ID NO:2, SEQ ID NO:3, SEQ ID NO:4, SEQ ID NO:5, SEQ ID NO:6, SEQ ID NO:7 or SEQ ID NO:8 or a functional analog thereof.

4. The isp-1 gene of claim 1 or 2, wherein said isp-1 gene has a sequence as set forth in SEQ ID NO:12 or codes for a protein having a sequence as set forth in SEQ ID NO:2.

5. An ISP-1 protein for use in altering a function at the level of cellular physiology involved in the regulation of developmental rates, behavioral rates, and longevity, wherein said ISP-1 protein is encoded by the gene of claim 1.

6. An ISP-1 protein for use in altering a function at the level of reactive oxygen species production, wherein said ISP-1 protein is encoded by the gene of claim 2.

7. Use of isp-1 gene to alter a function at the level of cellular physiology involved in the regulation of developmental rates, behavioral rates, and longevity in multicellular organisms, wherein isp-1 mutations cause a longer life and altered physiological rates relative to wild type.

8. Use of isp-1 gene to alter a function at the level of reactive oxygen species production in multicellular organisms, wherein isp-1 mutations cause a lower production of reactive oxygen species relative to wild type.

9. The use of claim 7 or 8, wherein said isp-1 gene has a sequence as set forth in SEQ ID NO:9, SEQ ID NO:10, SEQ ID NO:11 or SEQ ID NO:12, or an homologue thereof, wherein said homologue codes for a protein having a sequence as set forth in SEQ ID NO:1, SEQ ID NO:2, SEQ ID NO:3, SEQ ID NO:4, SEQ ID NO:5, SEQ ID NO:6, SEQ ID NO:7 or SEQ ID NO:8 or a functional analog thereof.

10. The use of claim 7 or 8, wherein said isp-1 gene has a sequence as set forth in SEQ ID NO:12 or codes for a protein having a sequence as set forth in SEQ ID NO:2.

11. Use of a ISP-1 protein to alter a function at the level of cellular physiology involved in the regulation of developmental rates, behavioral rates, and longevity, wherein said ISP-1 protein is encoded by a gene as defined in any one of claims 1, 3 or 34.

12. Use of a ISP-1 protein to alter a function at the level of reactive oxygen species production, wherein said ISP-1 protein is encoded by a gene as defined in any one of claims 1, 3 or 34.

13. A ctb-1 gene for use in altering a function at the level of cellular physiology involved in developmental rates, behavioral rates, and longevity, wherein ctb-1 mutations cause altered developmental and behavioral rates relative to the wild type.

14. A ctb-1 gene for use in altering a function at the level of reactive oxygen species production.

15. The ctb-1 gene of claim 13 or 14, wherein said ctb-1 gene has a sequence as set forth in SEQ ID NO:20 or SEQ ID NO:21, or an homologue thereof, wherein said homologue codes for a protein having sequence as set forth in SEQ ID NO:13, SEQ ID NO:14, SEQ ID NO:15, SEQ ID NO:16, SEQ ID NO:17, SEQ ID NO:18 or SEQ ID NO:19, or a functional analog thereof.

16. The ctb-1 gene of claim 13 or 14, wherein said ctb-1 gene has a sequence as set forth in SEQ ID NO:21 or codes for a protein having a sequence as set forth in SEQ ID NO:14.

17. A CTB-1 protein which has a function at the level of cellular physiology involved in the regulation of developmental rates, behavioral rates, and longevity, wherein said CTB-1 protein is encoded by the gene of claim 13.

18. A CTB-1 protein for use in altering a function at the level of reactive oxygen species production, wherein said CTB-1 protein is encoded by the gene of claim 14.

19. Use of ctb-1 gene to alter a function at the level of cellular physiology involved in the regulation of developmental rates, behavioral rates, and longevity in a multicellular organism, wherein ctb-1 mutations cause altered physiological rates relative to wild type.

20. Use of ctb-1 gene to alter a function at the level of reactive oxygen species production in a multicellular organism, wherein ctb-1 mutations cause an altered production of reactive oxygen species relative to wild type.

21. The use of claim 19 or 20, wherein said ctb-1 gene has a sequence as set forth in SEQ ID NO:20 or SEQ ID NO:21, or an homologue thereof, wherein said homologue codes for a protein having sequence as set forth in SEQ ID NO:13, SEQ ID NO:14, SEQ ID NO:15, SEQ ID NO:16, SEQ ID NO:17, SEQ ID NO:18 or SEQ ID NO:19, or a functional analog thereof.

22. The use of claim 19 or 20, wherein said ctb-1 gene has a sequence as set forth in SEQ ID NO:21 or codes for a protein having a sequence as set forth in SEQ ID NO:14.

23. Use of a CTB-1 protein to alter a function at the level of cellular physiology involved in the regulation of developmental rates, behavior rates, and longevity, wherein said CTB-1 protein is encoded by a gene as set forth in SEQ ID NO:13, SEQ ID NO:14, SEQ ID NO:15, SEQ ID NO:16, SEQ ID NO:17, SEQ ID NO:18, or SEQ ID NO:19, or an homologue thereof still coding for said CTB-1 protein.

24. Use of a CTB-1 protein to alter a function at the level of reactive oxygen species production, wherein said CTB-1 protein is encoded by a gene as set forth in SEQ ID NO:13, SEQ ID NO:14, SEQ ID NO:15, SEQ ID NO:16, SEQ ID NO:17, SEQ ID NO:18, or SEQ ID NO:19, or an homologue thereof still coding for said CTB-1 protein.

25. A method for screening a compound increasing developmental rates, behavioral rates, mitochondrial function, mitochondrial complex III function and/or increasing reactive oxygen species production, comprising the steps of: a) administering a compound to be screened to an isp-1 mutant organism, or isp-1; ctb-1 double mutant organism; and b) measuring developmental rates, behavioral rates, mitochondrial function, mitochondrial complex III function and/or production of reactive oxygen species of said mutant of step a), wherein an increase in developmental rates, behavioral rates, mitochondrial function, mitochondrial complex III function and/or an increased production of reactive oxygen species of said mutant organism, with respect to a wild-type organism is indicative of said compound being useful to manipulate cellular physiology and for therapeutic use in diseases states.

26. A method for screening a compound decreasing developmental rates, behavioral rates, mitochondrial function, mitochondrial complex III function and/or decreasing reactive oxygen species production, comprising the steps of: a) administering a compound to be screened to an organism, and b) measuring developmental rates, behavioral rates, mitochondrial function, mitochondrial complex III function and/or production of reactive oxygen species of said organism of step a) wherein a decrease in developmental rates, behavioral rates, mitochondrial function, mitochondrial complex III function and/or a decreased production of reactive oxygen species of said organism, with respect to control organism is indicative of said compound being useful to manipulate cellular physiology and increase longevity, and for therapeutic use in disease states.

27. The method of claim 26, wherein the disease states are selected from the group consisting of reactive oxygen species (ROS) mediated diseases, diabetes, hypoxia/reoxygenation injury and Parkinson's disease.

28. A method for screening a compound decreasing or increasing the function of ISP-1 or CTB-1 comprising the steps of: a) applying a compound to be screened to an in vitro preparation containing ISP-1 or CTB-1, and b) measuring ISP-1 or CTB-1 activity of said in vitro preparation of step a), wherein an increase or decrease of activity with respect to an untreated preparation is indicative of said compound being useful to manipulate cellular physiology, developmental rates, behavioral rates, reactive oxygen species production, longevity and for therapeutic use in disease states.

29. The method of claim 27, wherein the disease states are selected from the group consisting of reactive oxygen species (ROS) mediated diseases, diabetes, hypoxia/reoxygenation injury and Parkinson's disease.

30. A method to increase the life span of a multicellular organism, which comprises decreasing the activity of the isp-1 or ctb-1 genes.

31. Use of a compound for the manufacture of a medicament for increasing or decreasing physiological rate of tissues, organs, or whole organisms, wherein said compound is altering the activity of the genes isp-1 and/or ctb-1, and/or of ISP-1 and/or CTB-1.

32. Use of a compound for the manufacture of a medicament for increasing or decreasing physiological rate of tissues, organs, or whole organisms, wherein said compound is altering the activity of the genes isp-1 and/or ctb-1, and/or of ISP-1 and/or CTB-1, in a way that mimics the functional changes produced by the isp-1 (qm150) and/or ctb-1 (qm189) mutations.

33. A method for screening a compound increasing the function of complex III, isp-1, and/or replacing the function of ubiquinone in complex III, comprising the steps of: a) administrating a compound to be screened to an clk-1; lisp-1 double mutant organism; and b) assessing viability of said mutant organism of step a), wherein a viable mutant organism is indicative of said compound being useful for manipulating mitochondrial and cellular physiology and for therapeutic use in disease states.

34. The isp-1 gene of claim 2, wherein said isp-1 gene has a sequence as set forth in SEQ ID NO:9, SEQ ID NO:10, SEQ ID NO:11 or SEQ ID NO:12, or an homologue thereof, wherein said homologue codes for a protein having a sequence as set forth in SEQ ID NO:1, SEQ ID NO:2, SEQ ID NO:3, SEQ ID NO:4, SEQ ID NO:5, SEQ ID NO:6, SEQ ID NO:7 or SEQ ID NO:8 or a functional analog thereof.


SEQUENCE LISTING (27 sequences)

SEQ ID NO: 1 - protein, 276 aa, Caenorhabditis elegans
MASLARSGGFAVKVISGSQQHVNFVAATPAADNVAAFRDVPAEFSTAEARQAAVSHSTGGVFTNGLAVKGINVTSRRLAHTDVTFPDMSNYRRDSTKNTQVPARDTEDQRRALPTALYYGAGGVLSLWAGKEVVQTLVSYKAMAADQRALASIEINMADIPEGKTKTFEWRGKPVFVKHRTKAEIAKEKAVPVADLRHPQHDDERVQKDEWSVVIGVCTHLGCVPIADAGDYGGYYCPCHGSHYDASGRIRKGPAPLNLHVPAYSFKDATIVIGSS

SEQ ID NO: 2 - protein, 276 aa, Caenorhabditis elegans
MASLARSGGFAVKVISGSQQHVNFVAATPAADNVAAFRDVPAEFSTAEARQAAVSHSTGGVFTNGLAVKGINVTSRRLAHTDVTFPDMSNYRRDSTKNTQVPARDTEDQRRALPTALYYGAGGVLSLWAGKEVVQTLVSYKAMAADQRALASIEINMADIPEGKTKTFEWRGKPVFVKHRTKAEIAKEKAVPVADLRHPQHDDERVQKDEWSVVIGVCTHLGCVSIADAGDYGGYYCPCHGSHYDASGRIRKGPAPLNLHVPAYSFKDATIVIGSS

SEQ ID NO: 3 - protein, 229 aa, Arabidopsis thaliana
MASSSLSPATQLGSSRSALMAMSSGLFVKPTKMNHQMVRKEKIGLRISCQASSIPADRVPDMEKRKTLNLLLLGALSLPTGYMLVPYATFFVPPGTGGGGGGTPAKDALGNDVVAAEWLKTHGPGDRTLTQGLKGDPTYLVVENDKTLATYGINAVCTHLGCVVPWNKAENKFLCPCHGSQYNAQGRVVRGPAPLSLALAHADIDEAGKVLFVPWVETDFRTGDAPWWS

SEQ ID NO: 4 - protein, 196 aa, Gallus gallus
SHTDIKVPNFSDYRRPPDDYSTKSSRESDPSRKGFSYLVTAVTTLGVAYAAKNVVTQFVSSMSASADVLAMSKIEIKLSDIPEGKNMAFKWRGKPLFVRHRTKKEIDQEAAVEVSQLRDPQHDLERVKKPEWVILIGVCTHLGCVPIANAGDFGGYYCPCHGSHYDASGRIRKGPAPLNLEVPSYEFTSDDMVIVG

SEQ ID NO: 5 - protein, 215 aa, Saccharomyces cerevisiae
MLGIRSSVKTCFKPMSLTSKRLISQSLLASKSTYRTPNFDDVLKENNDADKGRSYAYFMVGAMGLLSSAGAKSTVETFISSMTATADVLAMAKVEVNLAAIPLGKNVVVKWQGKPVFIRHRTPHEIQEANSVDMSALKDPQTDADRVKDPQWLIMLGICTHLGCVPIGEAGDFGGWFCPCHGSHYDISGRIRKGPAPLNLEIPAYEFDGDKVIVG

SEQ ID NO: 6 - protein, 274 aa, Bos taurus
MLSVAARSGPFAPVLSATSRGVAGALRPLVQAAVPATSESPVLDLKRSVLCRESLRGQAAGRPLVASVSLNVPASVRYSHTDIKVPDFSDYRRPEVLDSTKSSKESSEARKGFSYLVTATTTVGVAYAAKNVVSQFVSSMSASADVLAMSKIEIKLSDIPEGKNMAFKWRGKPLFVRHRTKKEIDQEAAVEVSQLRDPQHDLERVKKPEWVILIGVCTHLGCVPIANAGDFGGYYCPCHGSHYDASGRIRKGPAPLNLEVPSYEFTSDDMVIVG

SEQ ID NO: 7 - protein, 274 aa, Homo sapiens
MLSVAARSGPFAPVLSATSRGVAGALRPLVQATVPATPEQPVLDLKRPFLSRESLSGQAVRRPLVASVGLNVPASVCYSHTDIKVPDFSEYRRLEVLDSTKSSRESSEARKGFSYLVTGVTTVGVAYAAKNAVTQFVSSMSASADVLALAKIEIKLSDIPEGKNMAFKWRGKPLFVRHRTQKEIEQEAAVELSQLRDPQHDLDRVKKPEWVILIGVCTHLGCVPIANAGDFGGYYCPCHGSHYDASGRIRLGPAPLNLEVPTYEFTSDDMVIVG

SEQ ID NO: 8 - protein, 256 aa, Rattus norvegicus
SRGVAGALRPLLQSAVPATSEPPVLDVKRPFLCRESLSGQAATRPLVATVGLNVPASVRYSHTDIKVPDFSDYRRAEVLDSTKSSKESSEARKGFSYLVTATTTVGVAYAAKNAVSQFVSSMSASADVLAMSKIEIKLSDIPEGKNMAFKWRGKPLFVRHRTKKEIDQEAAVEVSQLRDPQHDLERVKKPEWVILIGVCTHLGCVPIANAGDFGGYYCPCHGSHYDASGRIRKGPAPLNLEVPTYEFTSGDVVVVG

SEQ ID NO: 9 - DNA, 1885 bp, Caenorhabditis elegans
atggcttctcttgctagatctggaggttttgcagtcaaagtggtaagtattttttgaacaaatatgaagaaaaacactccaataatctattcaattcgagaaataatcatattttgcgagaaacgcgttcgaaaattttgtttaaaatgtatttgtttaaactattctcgttccaaaacactggacggtgtcgaaatgattaagatatcgattacagttctaaaattttgtccaatagtttataaatagttttctttcagatcagcggatcccagcaacatgtaaactttgttgccgcaacaccagccgccgataatgtaagtatttaaagttgcagaatattattttattgtctagtatccaggtcgctgctttccgcgatgttccagctgagttctccacggctgaagctcgtcaagccgctgttagtcacagcaccggaggtgtcttcaccaatggacttgctgtcaagggaatcaatggtatttgaaattgaatttaatatattaattcattaatttttaagtcacctctcgccgtttggctcacactgatgtgacattccccgatatgtcaaactatcgtcgtgattctacaaagaacactcaagtgccagcccgtgacaccgaggatcaacgtcgtgctcttccaactgctctgtattatggaggttcgaattattttttattcggtctaaacattgttttattacagctggaggagttctatctttgtgggccggaaaggaagttgtacagactcttgtttcttacaaagtatgttgaatttccattacaccgtttttaaattaatttttccaggctatggcagctgatcaacgtgccttggcttcgatcgaaatcaacatggctgatattccagaaggaaaaacaaagacattcgaatggcgtggaaagccagtcttcgtcaaacatcgtaccaaggctgagattgccaaggaaaaggccgtccctgtcgccgatctccgtcatccacaacatgacgacgaacgtgtgcagaaagatgaatggtccgttgtcattggagtgtgcacccatcttggatgtgtcccaattggtatgcagtcttttttctcctgaatttttgaaatcatattttgaattttcagccgatgctggagattacggaggttattactgcccttgtcacggatctcactacgacgcttctggacgtattagaaaggtaagacatttatttgctaaatgtagtacaatctgaaaaattcaaattttataattttttttcataatttattgaagaaaagttcgcgatttgatataattaatgataaattgtaattttctcttaaaattttataaaaagagagataatttaaacaaaaacaagaagatatcttctggaacgaacgaaagttcagtcctatcctaaccgttattacggtaaattttggtatttcttagtaaagcttttgataattttaaataaatacctagtttttacagtttctttttttttattaaaaaaaacggaataaaagagtagtaaataagttcgctttttttgatgaaaatcagtttttttccgaacgtaggaaaagtaattagacaattcttttttgaattcctaattatttacgtgaaaatctttatcataaattaagctctcagaatgttgaaaatttaaaaaaagttaaaacttcaatagcggaaaaaagtgtttatatttctaaatttgacattaaaaatgttttcaagtcaccctaacgttgataaaattcacaaaaatgtaaaggtgatctttttttcaaaatcttcattttttccagggaccagctccattgaatcttcacgtaccagcatactcgttcaaggatgccaccatcgtcattggatcgagctaa

SEQ ID NO: 10 - DNA, 1885 bp, Caenorhabditis elegans
atggcttctcttgctagatctggaggttttgcagtcaaagtggtaagtattttttgaacaaatatgaagaaaaacactccaataatctattcaattcgagaaataatcatattttgcgagaaacgcgttcgaaaattttgtttaaaatgtatttgtttaaactattctcgttccaaaacactggacggtgtcgaaatgattaagatatcgattacagttctaaaattttgtccaatagtttataaatagttttctttcagatcagcggatcccagcaacatgtaaactttgttgccgcaacaccagccgccgataatgtaagtatttaaagttgcagaatattattttattgtctagtatccaggtcgctgctttccgcgatgttccagctgagttctccacggctgaagctcgtcaagccgctgttagtcacagcaccggaggtgtcttcaccaatggacttgctgtcaagggaatcaatggtatttgaaattgaatttaatatattaattcattaatttttaagtcacctctcgccgtttggctcacactgatgtgacattccccgatatgtcaaactatcgtcgtgattctacaaagaacactcaagtgccagcccgtgacaccgaggatcaacgtcgtgctcttccaactgctctgtattatggaggttcgaattattttttattcggtctaaacattgttttattacagctggaggagttctatctttgtgggccggaaaggaagttgtacagactcttgtttcttacaaagtatgttgaatttccattacaccgtttttaaattaatttttccaggctatggcagctgatcaacgtgccttggcttcgatcgaaatcaacatggctgatattccagaaggaaaaacaaagacattcgaatggcgtggaaagccagtcttcgtcaaacatcgtaccaaggctgagattgccaaggaaaaggccgtccctgtcgccgatctccgtcatccacaacatgacgacgaacgtgtgcagaaagatgaatggtccgttgtcattggagtgtgcacccatcttggatgtgtctcaattggtatgcagtcttttttctcctgaatttttgaaatcatattttgaattttcagccgatgctggagattacggaggttattactgcccttgtcacggatctcactacgacgcttctggacgtattagaaaggtaagacatttatttgctaaatgtagtacaatctgaaaaattcaaattttataattttttttcataatttattgaagaaaagttcgcgatttgatataattaatgataaattgtaattttctcttaaaattttataaaaagagagataatttaaacaaaaacaagaagatatcttctggaacgaacgaaagttcagtcctatcctaaccgttattacggtaaattttggtatttcttagtaaagcttttgataattttaaataaatacctagtttttacagtttctttttttttattaaaaaaaacggaataaaagagtagtaaataagttcgctttttttgatgaaaatcagtttttttccgaacgtaggaaaagtaattagacaattcttttttgaattcctaattatttacgtgaaaatctttatcataaattaagctctcagaatgttgaaaatttaaaaaaagttaaaacttcaatagcggaaaaaagtgtttatatttctaaatttgacattaaaaatgttttcaagtcaccctaacgttgataaaattcacaaaaatgtaaaggtgatctttttttcaaaatcttcattttttccagggaccagctccattgaatcttcacgtaccagcatactcgttcaaggatgccaccatcgtcattggatcgagctaa

SEQ ID NO: 11 - DNA, 831 bp, Caenorhabditis elegans
atggcttctcttgctagatctggaggttttgcagtcaaagtgatcagcggatcccagcaacatgtaaactttgttgccgcaacaccagccgccgataatgtcgctgctttccgcgatgttccagctgagttctccacggctgaagctcgtcaagccgctgttagtcacagcaccggaggtgtcttcaccaatggacttgctgtcaagggaatcaatgtcacctctcgccgtttggctcacactgatgtgacattccccgatatgtcaaactatcgtcgtgattctacaaagaacactcaagtgccagcccgtgacaccgaggatcaacgtcgtgctcttccaactgctctgtattatggagctggaggagttctatctttgtgggccggaaaggaagttgtacagactcttgtttcttacaaagctatggcagctgatcaacgtgccttggcttcgatcgaaatcaacatggctgatattccagaaggaaaaacaaagacattcgaatggcgtggaaagccagtcttcgtcaaacatcgtaccaaggctgagattgccaaggaaaaggccgtccctgtcgccgatctccgtcatccacaacatgacgacgaacgtgtgcagaaagatgaatggtccgttgtcattggagtgtgcacccatcttggatgtgtcccaattgccgatgctggagattacggaggttattactgcccttgtcacggatctcactacgacgcttctggacgtattagaaagggaccagctccattgaatcttcacgtaccagcatactcgttcaaggatgccaccatcgtcattggatcgagctaa

SEQ ID NO: 12 - DNA, 831 bp, Caenorhabditis elegans
atggcttctcttgctagatctggaggttttgcagtcaaagtgatcagcggatcccagcaacatgtaaactttgttgccgcaacaccagccgccgataatgtcgctgctttccgcgatgttccagctgagttctccacggctgaagctcgtcaagccgctgttagtcacagcaccggaggtgtcttcaccaatggacttgctgtcaagggaatcaatgtcacctctcgccgtttggctcacactgatgtgacattccccgatatgtcaaactatcgtcgtgattctacaaagaacactcaagtgccagcccgtgacaccgaggatcaacgtcgtgctcttccaactgctctgtattatggagctggaggagttctatctttgtgggccggaaaggaagttgtacagactcttgtttcttacaaagctatggcagctgatcaacgtgccttggcttcgatcgaaatcaacatggctgatattccagaaggaaaaacaaagacattcgaatggcgtggaaagccagtcttcgtcaaacatcgtaccaaggctgagattgccaaggaaaaggccgtccctgtcgccgatctccgtcatccacaacatgacgacgaacgtgtgcagaaagatgaatggtccgttgtcattggagtgtgcacccatcttggatgtgtctcaattgccgatgctggagattacggaggttattactgcccttgtcacggatctcactacgacgcttctggacgtattagaaagggaccagctccattgaatcttcacgtaccagcatactcgttcaaggatgccaccatcgtcattggatcgagctaa

SEQ ID NO: 13 - protein, 370 aa, Caenorhabditis elegans
MKINNSLLNFVNGMLVTLPSSKTLTLSWNFGSMLGMVLIFQILTGTFLAFYYTPDSLMAFSTVQYIMYEVNFGWVFRIFHFNGASLFFIFLYLHIFKGLFFMSYRLKKVWMSGLTIYLLVMMEAFMGYVLVWAQMSFWAAVVITSLLSVIPIWGPTIVTWIWSGFGVTGATLKFFFVLHFLLPWAILVIVLGHLIFLHSTGSTSSLYCHGDYDKVCFSPEYLGKDAYNIVIWLLFIVLSLIYPFNLGDAEMFIEADPMMSPVHIVPEWYFLFAYAILRAIPNKVLGVIALLMSIVTFYFFALVNNYTSCLTKLNKFLVFMFIISSTILSWLGQCTVEDPFTILSPLFSFIYFGLAYLMLFIFMSSKLLFK

SEQ ID NO: 14 - protein, 370 aa, Caenorhabditis elegans
MKINNSLLNFVNGMLVTLPSSKTLTLSWNFGSMLGMVLIFQILTGTFLAFYYTPDSLMAFSTVQYIMYEVNFGWVFRIFHFNGASLFFIFLYLHIFKGLFFMSYRLKKVWMSGLTIYLLVMMEAFMGYVLVWAQMSFWAAVVITSLLSVIPIWGPTIVTWIWSGFGVTGVTLKFFFVLHFLLPWAILVIVLGHLIFLHSTGSTSSLYCHGDYDKVCFSPEYLGKDAYNIVIWLLFIVLSLIYPFNLGDAEMFIEADPMMSPVHIVPEWYFLFAYAILRAIPNKVLGVIALLMSIVTFYFFALVNNYTSCLTKLNKFLVFMFIISSTILSWLGQCTVEDPFTILSPLFSFIYFGLAYLMLFIFMSSKLLFK

SEQ ID NO: 15 - protein, 378 aa, Homo sapiens
MTPMRKTNPLMKLINHSFIDLPTPSNISAWWNFGSLLGACLILQITTGLFLAMHYSPDASTAFSSIAHITRDVNYGWIIRYLHANGASMFFICLFLHIGRGLYYGSFLYSETWNIGIILLLATMATAFMGYVLPWGQMSFWGATVITNLLSAIPYIGTDLVQWIWGGYSVDSPTLTRFFTFHFILPFIIAALATLHLLFLHETGSNNPLGITSHSDKITFHPYYTIKDALGLLLFLLSLMTLTLFSPDLLGDPDNYTLANPLNTPPHIKPEWYFLFAYTILRSVPNKLGGVLALLLSILILAMIPILHMSKQQSMMFRPLSQSLYWLLAADLLILTWIGGQPVSYPFTIIGQVASVLYFTTILILMPTISLIENKMLK

SEQ ID NO: 16 - protein, 215 aa, Arabidopsis thaliana
MSKVYDWFEERLEIQAIADDITSKYVPPHVNIFYCLGGITLTCFLVQVATGFAMTFYYRPTVTEAFASVQYIMTEANFGWLIRSVHRWSASMMVLMMILHVFRVYLTGGFKKPRELTWVTGVVLGVLTASFGVTGYSLPWDQIGYWAVKIVTGVPDAIPVIGSPLVELLRGSASVGQSTLTRFYSLHTFVLPLLTAVFMLMHFLMIRKQGISGPL

SEQ ID NO: 17 - protein, 379 aa, Bos taurus
MTNIRKSHPLMKIVNNAFIDLPAPSNISSWWNFGSLLGICLILQILTGLFLAMHYTSDTTTAFSSVTHICRDVNYGWIIRYMHANGASMFFICLYMHVGRGLYYGSYTFLETWNIGVILLLTVMATAFMGYVLPWGQMSFWGATVITNLLSAIPYIGTNLVEWIWGGFSVDKATLTRFFAFHFILPFIIMAIAMVHLLFLHETGSNNPTGISSDVDKIPFHPYYTIKDILGALLLILALMLLVLFAPDLLGDPDNYTPANPLNTPPHIKPEWYFLFAYAILRSIPNKLGGVLALAFSILILALIPLLHTSKQRSMMFRPLSQCLFWALVADLLTLTWIGGQPVEHPYITIGQLASVLYFLLILVLMPTAGTIENKLLKW

SEQ ID NO: 18 - protein, 385 aa, Saccharomyces cerevisiae
MAFRKSNVYLSLVNSYIIDSPQPSSINYWWNMGSLLGLCLVIQIVTGIFMAMHYSSNIELAFSSVEHIMRDVHNGYILRYLHANGASFFFMVMFMHMAKGLYYGSYRSPRVTLWNVGVIIFILTIATAFLGYCCVYGQMSHWGATVITNLFSAIPFVGNDIVSWLWGGFSVSNPTIQRFFALHYLVPFIIAAMVIMHLMALHIHGSSNPLGITGNLDRIPMHSYFIFKDLVTVFLFMLILALFVFYSPNTLGHPDNYIPGNPLVTPASIVPEWYLLPFYAILRSIPDKLLGVITMFAAILVLLVLPFTDRSVVRGNTFKVLSKFFFFIFVFNFVLLGQIGACHVEVPYVLMGQIATFIYFAYFLIIVPVISTIENVLFYIGRVNK

SEQ ID NO: 19 - protein, 380 aa, Gallus gallus
MAPNIRKSHPLLKMINNSLIDLPAPSNISAWWNFGSLLAVCLMTQILTGLLLAMHYTADTSLAFSSVAHTCRNVQYGWLIRNLHANGASFFFICIFLHIGRGLYYGSYLYKETWNTGVILLLTLMATAFVGYVLPWGQMSFWGATVITNLFSAIPYIGHTLVEWAWGGFSVDNPTLTRFFALHFLLPFAIAGITIIHLTFLHESGSNNPLGISSDSDKIPFHPYYSFKDILGLTLMLTPFLTLALFSPNLLGDPENFTPANPLVTPPHIKPEWYFLFAYAILRSIPNKLGGVLALAASVLILFLIPFLHKSKQRTMTFRPLSQTLFWLLVANLLILTWIGSQPVEHPFIIIGQMASLSYFTILLILFPTIGTLENKMLNY

SEQ ID NO: 20 - DNA, 1113 bp, Caenorhabditis elegans
ttgaaaattaataatagattattaaattttgttaatgggatgttggtgacattgccatctagaaaaactttaacattaagatgaaattttggtagtatattgggtatagttttaatctttcagattttaacaggtacatttttagcattttattatacgcccgataggttaatagcattttcaacagtgcagtatattatgtatgaggtaaattttggatgagtatttcgaatttttcattttaatggggccaggttattttttatttttttgtatttacatatttttaaagggttattttttataagatatcgtttaaaaaaagtatgaatgtctggtttaacaatttatttattagtaataatagaagcttttataggttatgttttagtttgagctcaaataagattttgagcagcagtagttattactagacttttaagagttattccaatttgagggccaactattgttacttgaatttgaagaggttttggtgttacaggggcaacattaaaattcttttttgtattacattttttattgccgtgagctattctagttattgtattggggcatttaatttttttacacagaactggtagaacatctaggttatattgccacggtgattatgataaagtttgttttagacctgagtacttaggtaaagatgcttataatattgttatttgattattatttattgtgttaaggttgatttacccgtttaatttaggtgatgcagagatgtttattgaagctgaccctataatgaggccagttcatattgttccagagtgatattttttatttgcttatgcaattttgcgtgctattccaaataaagtcttaggggtaattgctttattaataaggatcgtaacattttatttttttgctttagttaataattatacctcttgtttaaccaaattaaataaatttttagtatttatatttattatctcatctactattttaagatgactaggtcaatgcactgtagaagacccttttacaattttaagacctttattttcttttatttattttggtttagcttatttaatattgtttatttttataagaagtaaattgttatttaaataa

SEQ ID NO: 21 - DNA, 1113 bp, Caenorhabditis elegans
ttgaaaattaataatagattattaaattttgttaatgggatgttggtgacattgccatctagaaaaactttaacattaagatgaaattttggtagtatattgggtatagttttaatctttcagattttaacaggtacatttttagcattttattatacgcccgataggttaatagcattttcaacagtgcagtatattatgtatgaggtaaattttggatgagtatttcgaatttttcattttaatggggccaggttattttttatttttttgtatttacatatttttaaagggttattttttataagatatcgtttaaaaaaagtatgaatgtctggtttaacaatttatttattagtaataatagaagcttttataggttatgttttagtttgagctcaaataagattttgagcagcagtagttattactagacttttaagagttattccaatttgagggccaactattgttacttgaatttgaagaggttttggtgttacaggggtaacattaaaattcttttttgtattacattttttattgccgtgagctattctagttattgtattggggcatttaatttttttacacagaactggtagaacatctaggttatattgccacggtgattatgataaagtttgttttagacctgagtacttaggtaaagatgcttataatattgttatttgattattatttattgtgttaaggttgatttacccgtttaatttaggtgatgcagagatgtttattgaagctgaccctataatgaggccagttcatattgttccagagtgatattttttatttgcttatgcaattttgcgtgctattccaaataaagtcttaggggtaattgctttattaataaggatcgtaacattttatttttttgctttagttaataattatacctcttgtttaaccaaattaaataaatttttagtatttatatttattatctcatctactattttaagatgactaggtcaatgcactgtagaagacccttttacaattttaagacctttattttcttttatttattttggtttagcttatttaatattgtttatttttataagaagtaaattgttatttaaataa

SEQ ID NO: 22 - DNA, 24 bp, Artificial Sequence
  Primer SHP-1587
gctccgcctcatctagagaacctc

SEQ ID NO: 23 - DNA, 24 bp, Artificial Sequence
  Primer SHP-1588
cagtccgcccgttgagtttgtccc

SEQ ID NO: 24 - DNA, 25 bp, Artificial Sequence
  Primer SHP-1622
ccctgaagaggctaagaatattagg

SEQ ID NO: 25 - DNA, 28 bp, Artificial Sequence
  Primer SHP-1633
caatacaataactagaatagctcacggc

SEQ ID NO: 26 - DNA, 23 bp, Artificial Sequence
  Primer SHP-1623
gatcttaacattccggctgaggc

SEQ ID NO: 27 - DNA, 20 bp, Artificial Sequence
  Primer SHP-1632
ggttttggtgttacaggggc